SEQUENCE LISTING (594 sequences)

SEQ ID NO: 1 - protein, 5 aa, Bothrops jararaca
  mat_peptide (1): Xaa is a pyrrolidone carboxilic acid
XSWPP

SEQ ID NO: 2 - protein, 5 aa, Bothrops jararaca
  mat_peptide (1): Xaa is a pyrrolidone carboxilic acid
XKWPP

SEQ ID NO: 3 - protein, 5 aa, Bothrops jararaca
  mat_peptide (1): Xaa is a pyrrolidone carboxilic acid
XWFPP

SEQ ID NO: 4 - protein, 5 aa, Bothrops jararaca
  mat_peptide (1): Xaa is a pyrrolidone carboxilic acid
XSFPP

SEQ ID NO: 5 - protein, 5 aa, Bothrops jararaca
  mat_peptide (1): Xaa is a pyrrolidone carboxilic acid
XKFPP

SEQ ID NO: 6 - protein, 5 aa, Bothrops jararaca
  mat_peptide (1): Xaa is a pyrrolidone carboxilic acid
XWGPP

SEQ ID NO: 7 - protein, 5 aa, Bothrops jararaca
  mat_peptide (1): Xaa is a pyrrolidone carboxilic acid
XSGPP

SEQ ID NO: 8 - protein, 5 aa, Bothrops jararaca
  mat_peptide (1): Xaa is a pyrrolidone carboxilic acid
XKGPP

SEQ ID NO: 9 - protein, 5 aa, Bothrops jararaca
  mat_peptide (1): Xaa is a pyrrolidone carboxilic acid
XSWAP

SEQ ID NO: 10 - protein, 5 aa, Bothrops jararaca
  mat_peptide (1): Xaa is a pyrrolidone carboxilic acid
XKWAP

SEQ ID NO: 11 - protein, 5 aa, Bothrops jararaca
  mat_peptide (1): Xaa is a pyrrolidone carboxilic acid
XWPAP

SEQ ID NO: 12 - protein, 5 aa, Bothrops jararaca
  mat_peptide (1): Xaa is a pyrrolidone carboxilic acid
XSPAP

SEQ ID NO: 13 - protein, 5 aa, Bothrops jararaca
  mat_peptide (1): Xaa is a pyrrolidone carboxilic acid
XKPAP

SEQ ID NO: 14 - protein, 5 aa, Bothrops jararaca
  mat_peptide (1): Xaa is a pyrrolidone carboxilic acid
XWFAP

SEQ ID NO: 15 - protein, 5 aa, Bothrops jararaca
  mat_peptide (1): Xaa is a pyrrolidone carboxilic acid
XSFAP

SEQ ID NO: 16 - protein, 5 aa, Bothrops jararaca
  mat_peptide (1): Xaa is a pyrrolidone carboxilic acid
XKFAP

SEQ ID NO: 17 - protein, 5 aa, Bothrops jararaca
  mat_peptide (1): Xaa is a pyrrolidone carboxilic acid
XWGAP

SEQ ID NO: 18 - protein, 5 aa, Bothrops jararaca
  mat_peptide (1): Xaa is a pyrrolidone carboxilic acid
XSGAP

SEQ ID NO: 19 - protein, 5 aa, Bothrops jararaca
  mat_peptide (1): Xaa is a pyrrolidone carboxilic acid
XKGAP

SEQ ID NO: 20 - protein, 5 aa, Bothrops jararaca
  mat_peptide (1): Xaa is a pyrrolidone carboxilic acid
XSWRP

SEQ ID NO: 21 - protein, 5 aa, Bothrops jararaca
  mat_peptide (1): Xaa is a pyrrolidone carboxilic acid
XKWRP

SEQ ID NO: 22 - protein, 5 aa, Bothrops jararaca
  mat_peptide (1): Xaa is a pyrrolidone carboxilic acid
XWPRP

SEQ ID NO: 23 - protein, 5 aa, Bothrops jararaca
  mat_peptide (1): Xaa is a pyrrolidone carboxilic acid
XSPRP

SEQ ID NO: 24 - protein, 5 aa, Bothrops jararaca
  mat_peptide (1): Xaa is a pyrrolidone carboxilic acid
XKPRP

SEQ ID NO: 25 - protein, 5 aa, Bothrops jararaca
  mat_peptide (1): Xaa is a pyrrolidone carboxilic acid
XWFRP

SEQ ID NO: 26 - protein, 5 aa, Bothrops jararaca
  mat_peptide (1): Xaa is a pyrrolidone carboxilic acid
XSFRP

SEQ ID NO: 27 - protein, 5 aa, Bothrops jararaca
  mat_peptide (1): Xaa is a pyrrolidone carboxilic acid
XKFRP

SEQ ID NO: 28 - protein, 5 aa, Bothrops jararaca
  mat_peptide (1): Xaa is a pyrrolidone carboxilic acid
XWGRP

SEQ ID NO: 29 - protein, 5 aa, Bothrops jararaca
  mat_peptide (1): Xaa is a pyrrolidone carboxilic acid
XSGRP

SEQ ID NO: 30 - protein, 5 aa, Bothrops jararaca
  mat_peptide (1): Xaa is a pyrrolidone carboxilic acid
XKGRP

SEQ ID NO: 31 - protein, 5 aa, Bothrops jararaca
  mat_peptide (1): Xaa is a pyrrolidone carboxilic acid
XSWXP

SEQ ID NO: 32 - protein, 5 aa, Bothrops jararaca
  mat_peptide (1): Xaa is a pyrrolidone carboxilic acid
XKWXP

SEQ ID NO: 33 - protein, 5 aa, Bothrops jararaca
  mat_peptide (1): Xaa is a pyrrolidone carboxilic acid
XWPXP

SEQ ID NO: 34 - protein, 5 aa, Bothrops jararaca
  mat_peptide (1): Xaa is a pyrrolidone carboxilic acid
XSPXP

SEQ ID NO: 35 - protein, 5 aa, Bothrops jararaca
  mat_peptide (1): Xaa is a pyrrolidone carboxilic acid
XKPXP

SEQ ID NO: 36 - protein, 5 aa, Bothrops jararaca
  mat_peptide (1): Xaa is a pyrrolidone carboxilic acid
XWFXP

SEQ ID NO: 37 - protein, 5 aa, Bothrops jararaca
  mat_peptide (1): Xaa is a pyrrolidone carboxilic acid
XSFXP

SEQ ID NO: 38 - protein, 5 aa, Bothrops jararaca
  mat_peptide (1): Xaa is a pyrrolidone carboxilic acid
XKFXP

SEQ ID NO: 39 - protein, 5 aa, Bothrops jararaca
  mat_peptide (1): Xaa is a pyrrolidone carboxilic acid
XWGXP

SEQ ID NO: 40 - protein, 5 aa, Bothrops jararaca
  mat_peptide (1): Xaa is a pyrrolidone carboxilic acid
XSGXP

SEQ ID NO: 41 - protein, 5 aa, Bothrops jararaca
  mat_peptide (1): Xaa is a pyrrolidone carboxilic acid
XKGXP

SEQ ID NO: 42 - protein, 5 aa, Bothrops jararaca
  mat_peptide (1): Xaa is a pyrrolidone carboxilic acid
XSWPX

SEQ ID NO: 43 - protein, 5 aa, Bothrops jararaca
  mat_peptide (1): Xaa is a pyrrolidone carboxilic acid
XKWPX

SEQ ID NO: 44 - protein, 5 aa, Bothrops jararaca
  mat_peptide (1): Xaa is a pyrrolidone carboxilic acid
XWFPX

SEQ ID NO: 45 - protein, 5 aa, Bothrops jararaca
  mat_peptide (1): Xaa is a pyrrolidone carboxilic acid
XSFPX

SEQ ID NO: 46 - protein, 5 aa, Bothrops jararaca
  mat_peptide (1): Xaa is a pyrrolidone carboxilic acid
XKFPX

SEQ ID NO: 47 - protein, 5 aa, Bothrops jararaca
  mat_peptide (1): Xaa is a pyrrolidone carboxilic acid
XWGPX

SEQ ID NO: 48 - protein, 5 aa, Bothrops jararaca
  mat_peptide (1): Xaa is a pyrrolidone carboxilic acid
XSGPX

SEQ ID NO: 49 - protein, 5 aa, Bothrops jararaca
  mat_peptide (1): Xaa is a pyrrolidone carboxilic acid
XKGPX

SEQ ID NO: 50 - protein, 5 aa, Bothrops jararaca
  mat_peptide (1): Xaa is a pyrrolidone carboxilic acid
XSWAX

SEQ ID NO: 51 - protein, 5 aa, Bothrops jararaca
  mat_peptide (1): Xaa is a pyrrolidone carboxilic acid
XKWAX

SEQ ID NO: 52 - protein, 5 aa, Bothrops jararaca
  mat_peptide (1): Xaa is a pyrrolidone carboxilic acid
XWPAX

SEQ ID NO: 53 - protein, 5 aa, Bothrops jararaca
  mat_peptide (1): Xaa is a pyrrolidone carboxilic acid
XSPAX

SEQ ID NO: 54 - protein, 5 aa, Bothrops jararaca
  mat_peptide (1): Xaa is a pyrrolidone carboxilic acid
XKPAX

SEQ ID NO: 55 - protein, 5 aa, Bothrops jararaca
  mat_peptide (1): Xaa is a pyrrolidone carboxilic acid
XWFAX

SEQ ID NO: 56 - protein, 5 aa, Bothrops jararaca
  mat_peptide (1): Xaa is a pyrrolidone carboxilic acid
XSFAX

SEQ ID NO: 57 - protein, 5 aa, Bothrops jararaca
  mat_peptide (1): Xaa is a pyrrolidone carboxilic acid
XKFAX

SEQ ID NO: 58 - protein, 5 aa, Bothrops jararaca
  mat_peptide (1): Xaa is a pyrrolidone carboxilic acid
XWGAX

SEQ ID NO: 59 - protein, 5 aa, Bothrops jararaca
  mat_peptide (1): Xaa is a pyrrolidone carboxilic acid
XSGAX

SEQ ID NO: 60 - protein, 5 aa, Bothrops jararaca
  mat_peptide (1): Xaa is a pyrrolidone carboxilic acid
XKGAX

SEQ ID NO: 61 - protein, 5 aa, Bothrops jararaca
  mat_peptide (1): Xaa is a pyrrolidone carboxilic acid
XSWRX

SEQ ID NO: 62 - protein, 5 aa, Bothrops jararaca
  mat_peptide (1): Xaa is a pyrrolidone carboxilic acid
XKWRX

SEQ ID NO: 63 - protein, 5 aa, Bothrops jararaca
  mat_peptide (1): Xaa is a pyrrolidone carboxilic acid
XWPRX

SEQ ID NO: 64 - protein, 5 aa, Bothrops jararaca
  mat_peptide (1): Xaa is a pyrrolidone carboxilic acid
XSPRX

SEQ ID NO: 65 - protein, 5 aa, Bothrops jararaca
  mat_peptide (1): Xaa is a pyrrolidone carboxilic acid
XKPRX

SEQ ID NO: 66 - protein, 5 aa, Bothrops jararaca
  mat_peptide (1): Xaa is a pyrrolidone carboxilic acid
XWFRX

SEQ ID NO: 67 - protein, 5 aa, Bothrops jararaca
  mat_peptide (1): Xaa is a pyrrolidone carboxilic acid
XSFRX

SEQ ID NO: 68 - protein, 5 aa, Bothrops jararaca
  mat_peptide (1): Xaa is a pyrrolidone carboxilic acid
XKFRX

SEQ ID NO: 69 - protein, 5 aa, Bothrops jararaca
  mat_peptide (1): Xaa is a pyrrolidone carboxilic acid
XWGRX

SEQ ID NO: 70 - protein, 5 aa, Bothrops jararaca
  mat_peptide (1): Xaa is a pyrrolidone carboxilic acid
XSGRX

SEQ ID NO: 71 - protein, 5 aa, Bothrops jararaca
  mat_peptide (1): Xaa is a pyrrolidone carboxilic acid
XKGRX

SEQ ID NO: 72 - protein, 5 aa, Bothrops jararaca
  mat_peptide (1): Xaa is a pyrrolidone carboxilic acid
XSWPX

SEQ ID NO: 73 - protein, 5 aa, Bothrops jararaca
  mat_peptide (1): Xaa is a pyrrolidone carboxilic acid
XKWPX

SEQ ID NO: 74 - protein, 5 aa, Bothrops jararaca
  mat_peptide (1): Xaa is a pyrrolidone carboxilic acid
XWFPX

SEQ ID NO: 75 - protein, 5 aa, Bothrops jararaca
  mat_peptide (1): Xaa is a pyrrolidone carboxilic acid
XSFPX

SEQ ID NO: 76 - protein, 5 aa, Bothrops jararaca
  mat_peptide (1): Xaa is a pyrrolidone carboxilic acid
XKFPX

SEQ ID NO: 77 - protein, 5 aa, Bothrops jararaca
  mat_peptide (1): Xaa is a pyrrolidone carboxilic acid
XWGPX

SEQ ID NO: 78 - protein, 5 aa, Bothrops jararaca
  mat_peptide (1): Xaa is a pyrrolidone carboxilic acid
XSGPX

SEQ ID NO: 79 - protein, 5 aa, Bothrops jararaca
  mat_peptide (1): Xaa is a pyrrolidone carboxilic acid
XKGPX

SEQ ID NO: 80 - protein, 5 aa, Bothrops jararaca
  mat_peptide (1): Xaa is a pyrrolidone carboxilic acid
XWWAX

SEQ ID NO: 81 - protein, 5 aa, Bothrops jararaca
  mat_peptide (1): Xaa is a pyrrolidone carboxilic acid
XSWAX

SEQ ID NO: 82 - protein, 5 aa, Bothrops jararaca
  mat_peptide (1): Xaa is a pyrrolidone carboxilic acid
XKWAX

SEQ ID NO: 83 - protein, 5 aa, Bothrops jararaca
  mat_peptide (1): Xaa is a pyrrolidone carboxilic acid
XWPAX

SEQ ID NO: 84 - protein, 5 aa, Bothrops jararaca
  mat_peptide (1): Xaa is a pyrrolidone carboxilic acid
XSPAX

SEQ ID NO: 85 - protein, 5 aa, Bothrops jararaca
  mat_peptide (1): Xaa is a pyrrolidone carboxilic acid
XKPAX

SEQ ID NO: 86 - protein, 5 aa, Bothrops jararaca
  mat_peptide (1): Xaa is a pyrrolidone carboxilic acid
XWFAX

SEQ ID NO: 87 - protein, 5 aa, Bothrops jararaca
  mat_peptide (1): Xaa is a pyrrolidone carboxilic acid
XSFAX

SEQ ID NO: 88 - protein, 5 aa, Bothrops jararaca
  mat_peptide (1): Xaa is a pyrrolidone carboxilic acid
XKFAX

SEQ ID NO: 89 - protein, 5 aa, Bothrops jararaca
  mat_peptide (1): Xaa is a pyrrolidone carboxilic acid
XWGAX

SEQ ID NO: 90 - protein, 5 aa, Bothrops jararaca
  mat_peptide (1): Xaa is a pyrrolidone carboxilic acid
XSGAX

SEQ ID NO: 91 - protein, 5 aa, Bothrops jararaca
  mat_peptide (1): Xaa is a pyrrolidone carboxilic acid
XKGAX

SEQ ID NO: 92 - protein, 5 aa, Bothrops jararaca
  mat_peptide (1): Xaa is a pyrrolidone carboxilic acid
XWWRX

SEQ ID NO: 93 - protein, 5 aa, Bothrops jararaca
  mat_peptide (1): Xaa is a pyrrolidone carboxilic acid
XSWRX

SEQ ID NO: 94 - protein, 5 aa, Bothrops jararaca
  mat_peptide (1): Xaa is a pyrrolidone carboxilic acid
XKWRX

SEQ ID NO: 95 - protein, 5 aa, Bothrops jararaca
  mat_peptide (1): Xaa is a pyrrolidone carboxilic acid
XWPRX

SEQ ID NO: 96 - protein, 5 aa, Bothrops jararaca
  mat_peptide (1): Xaa is a pyrrolidone carboxilic acid
XSPRX

SEQ ID NO: 97 - protein, 5 aa, Bothrops jararaca
  mat_peptide (1): Xaa is a pyrrolidone carboxilic acid
XKPRX

SEQ ID NO: 98 - protein, 5 aa, Bothrops jararaca
  mat_peptide (1): Xaa is a pyrrolidone carboxilic acid
XWFRX

SEQ ID NO: 99 - protein, 5 aa, Bothrops jararaca
  mat_peptide (1): Xaa is a pyrrolidone carboxilic acid
XSFRX

SEQ ID NO: 100 - protein, 5 aa, Bothrops jararaca
  mat_peptide (1): Xaa is a pyrrolidone carboxilic acid
XKFRX

SEQ ID NO: 101 - protein, 5 aa, Bothrops jararaca
  mat_peptide (1): Xaa is a pyrrolidone carboxilic acid
XWGRX

SEQ ID NO: 102 - protein, 5 aa, Bothrops jararaca
  mat_peptide (1): Xaa is a pyrrolidone carboxilic acid
XSGRX

SEQ ID NO: 103 - protein, 5 aa, Bothrops jararaca
  mat_peptide (1): Xaa is a pyrrolidone carboxilic acid
XKGRX

SEQ ID NO: 104 - protein, 6 aa, Bothrops jararaca
  mat_peptide (1): Xaa is a pyrrolidone carboxilic acid
XSWPRP

SEQ ID NO: 105 - protein, 6 aa, Bothrops jararaca
  mat_peptide (1): Xaa is a pyrrolidone carboxilic acid
XKWPRP

SEQ ID NO: 106 - protein, 6 aa, Bothrops jararaca
  mat_peptide (1): Xaa is a pyrrolidone carboxilic acid
XWFPRP

SEQ ID NO: 107 - protein, 6 aa, Bothrops jararaca
  mat_peptide (1): Xaa is a pyrrolidone carboxilic acid
XSFPRP

SEQ ID NO: 108 - protein, 6 aa, Bothrops jararaca
  mat_peptide (1): Xaa is a pyrrolidone carboxilic acid
XKFPRP

SEQ ID NO: 109 - protein, 6 aa, Bothrops jararaca
  mat_peptide (1): Xaa is a pyrrolidone carboxilic acid
XWGPRP

SEQ ID NO: 110 - protein, 6 aa, Bothrops jararaca
  mat_peptide (1): Xaa is a pyrrolidone carboxilic acid
XSGPRP

SEQ ID NO: 111 - protein, 6 aa, Bothrops jararaca
  mat_peptide (1): Xaa is a pyrrolidone carboxilic acid
XKGPRP

SEQ ID NO: 112 - protein, 6 aa, Bothrops jararaca
  mat_peptide (1): Xaa is a pyrrolidone carboxilic acid
XSWARP

SEQ ID NO: 113 - protein, 6 aa, Bothrops jararaca
  mat_peptide (1): Xaa is a pyrrolidone carboxilic acid
XKWARP

SEQ ID NO: 114 - protein, 6 aa, Bothrops jararaca
  mat_peptide (1): Xaa is a pyrrolidone carboxilic acid
XWPARP

SEQ ID NO: 115 - protein, 6 aa, Bothrops jararaca
  mat_peptide (1): Xaa is a pyrrolidone carboxilic acid
XSPARP

SEQ ID NO: 116 - protein, 6 aa, Bothrops jararaca
  mat_peptide (1): Xaa is a pyrrolidone carboxilic acid
XKPARP

SEQ ID NO: 117 - protein, 6 aa, Bothrops jararaca
  mat_peptide (1): Xaa is a pyrrolidone carboxilic acid
XWFARP

SEQ ID NO: 118 - protein, 6 aa, Bothrops jararaca
  mat_peptide (1): Xaa is a pyrrolidone carboxilic acid
XSFARP

SEQ ID NO: 119 - protein, 6 aa, Bothrops jararaca
  mat_peptide (1): Xaa is a pyrrolidone carboxilic acid
XKFARP

SEQ ID NO: 120 - protein, 6 aa, Bothrops jararaca
  mat_peptide (1): Xaa is a pyrrolidone carboxilic acid
XWGARP

SEQ ID NO: 121 - protein, 6 aa, Bothrops jararaca
  mat_peptide (1): Xaa is a pyrrolidone carboxilic acid
XSGARP

SEQ ID NO: 122 - protein, 6 aa, Bothrops jararaca
  mat_peptide (1): Xaa is a pyrrolidone carboxilic acid
XKGARP

SEQ ID NO: 123 - protein, 6 aa, Bothrops jararaca
  mat_peptide (1): Xaa is a pyrrolidone carboxilic acid
XSWIRP

SEQ ID NO: 124 - protein, 6 aa, Bothrops jararaca
  mat_peptide (1): Xaa is a pyrrolidone carboxilic acid
XKWIRP

SEQ ID NO: 125 - protein, 6 aa, Bothrops jararaca
  mat_peptide (1): Xaa is a pyrrolidone carboxilic acid
XWPIRP

SEQ ID NO: 126 - protein, 6 aa, Bothrops jararaca
  mat_peptide (1): Xaa is a pyrrolidone carboxilic acid
XSPIRP

SEQ ID NO: 127 - protein, 6 aa, Bothrops jararaca
  mat_peptide (1): Xaa is a pyrrolidone carboxilic acid
XKPIRP

SEQ ID NO: 128 - protein, 6 aa, Bothrops jararaca
  mat_peptide (1): Xaa is a pyrrolidone carboxilic acid
XWFIRP

SEQ ID NO: 129 - protein, 6 aa, Bothrops jararaca
  mat_peptide (1): Xaa is a pyrrolidone carboxilic acid
XSFIRP

SEQ ID NO: 130 - protein, 6 aa, Bothrops jararaca
  mat_peptide (1): Xaa is a pyrrolidone carboxilic acid
XKFIRP

SEQ ID NO: 131 - protein, 6 aa, Bothrops jararaca
  mat_peptide (1): Xaa is a pyrrolidone carboxilic acid
XWGIRP

SEQ ID NO: 132 - protein, 6 aa, Bothrops jararaca
  mat_peptide (1): Xaa is a pyrrolidone carboxilic acid
XSGIRP

SEQ ID NO: 133 - protein, 6 aa, Bothrops jararaca
  mat_peptide (1): Xaa is a pyrrolidone carboxilic acid
XKGIRP

SEQ ID NO: 134 - protein, 6 aa, Bothrops jararaca
  mat_peptide (1): Xaa is a pyrrolidone carboxilic acid
XSWPIP

SEQ ID NO: 135 - protein, 6 aa, Bothrops jararaca
  mat_peptide (1): Xaa is a pyrrolidone carboxilic acid
XKWPIP

SEQ ID NO: 136 - protein, 6 aa, Bothrops jararaca
  mat_peptide (1): Xaa is a pyrrolidone carboxilic acid
XWPPIP

SEQ ID NO: 137 - protein, 6 aa, Bothrops jararaca
  mat_peptide (1): Xaa is a pyrrolidone carboxilic acid
XSPPIP

SEQ ID NO: 138 - protein, 6 aa, Bothrops jararaca
  mat_peptide (1): Xaa is a pyrrolidone carboxilic acid
XKPPIP

SEQ ID NO: 139 - protein, 6 aa, Bothrops jararaca
  mat_peptide (1): Xaa is a pyrrolidone carboxilic acid
XWFPIP

SEQ ID NO: 140 - protein, 6 aa, Bothrops jararaca
  mat_peptide (1): Xaa is a pyrrolidone carboxilic acid
XSFPIP

SEQ ID NO: 141 - protein, 6 aa, Bothrops jararaca
  mat_peptide (1): Xaa is a pyrrolidone carboxilic acid
XKFPIP

SEQ ID NO: 142 - protein, 6 aa, Bothrops jararaca
  mat_peptide (1): Xaa is a pyrrolidone carboxilic acid
XWGPIP

SEQ ID NO: 143 - protein, 6 aa, Bothrops jararaca
  mat_peptide (1): Xaa is a pyrrolidone carboxilic acid
XSGPIP

SEQ ID NO: 144 - protein, 6 aa, Bothrops jararaca
  mat_peptide (1): Xaa is a pyrrolidone carboxilic acid
XKGPIP

SEQ ID NO: 145 - protein, 6 aa, Bothrops jararaca
  mat_peptide (1): Xaa is a pyrrolidone carboxilic acid
XSWAIP

SEQ ID NO: 146 - protein, 6 aa, Bothrops jararaca
  mat_peptide (1): Xaa is a pyrrolidone carboxilic acid
XKWAIP

SEQ ID NO: 147 - protein, 6 aa, Bothrops jararaca
  mat_peptide (1): Xaa is a pyrrolidone carboxilic acid
XWPAIP

SEQ ID NO: 148 - protein, 6 aa, Bothrops jararaca
  mat_peptide (1): Xaa is a pyrrolidone carboxilic acid
XSPAIP

SEQ ID NO: 149 - protein, 6 aa, Bothrops jararaca
  mat_peptide (1): Xaa is a pyrrolidone carboxilic acid
XKPAIP

SEQ ID NO: 150 - protein, 6 aa, Bothrops jararaca
  mat_peptide (1): Xaa is a pyrrolidone carboxilic acid
XWFAIP

SEQ ID NO: 151 - protein, 6 aa, Bothrops jararaca
  mat_peptide (1): Xaa is a pyrrolidone carboxilic acid
XSFAIP

SEQ ID NO: 152 - protein, 6 aa, Bothrops jararaca
  mat_peptide (1): Xaa is a pyrrolidone carboxilic acid
XKFAIP

SEQ ID NO: 153 - protein, 6 aa, Bothrops jararaca
  mat_peptide (1): Xaa is a pyrrolidone carboxilic acid
XWGAIP

SEQ ID NO: 154 - protein, 6 aa, Bothrops jararaca
  mat_peptide (1): Xaa is a pyrrolidone carboxilic acid
XSGAIP

SEQ ID NO: 155 - protein, 6 aa, Bothrops jararaca
  mat_peptide (1): Xaa is a pyrrolidone carboxilic acid
XKGAIP

SEQ ID NO: 156 - protein, 6 aa, Bothrops jararaca
  mat_peptide (1): Xaa is a pyrrolidone carboxilic acid
XSWRIP

SEQ ID NO: 157 - protein, 6 aa, Bothrops jararaca
  mat_peptide (1): Xaa is a pyrrolidone carboxilic acid
XKWRIP

SEQ ID NO: 158 - protein, 6 aa, Bothrops jararaca
  mat_peptide (1): Xaa is a pyrrolidone carboxilic acid
XWPRIP

SEQ ID NO: 159 - protein, 6 aa, Bothrops jararaca
  mat_peptide (1): Xaa is a pyrrolidone carboxilic acid
XSPRIP

SEQ ID NO: 160 - protein, 6 aa, Bothrops jararaca
  mat_peptide (1): Xaa is a pyrrolidone carboxilic acid
XKPRIP

SEQ ID NO: 161 - protein, 6 aa, Bothrops jararaca
  mat_peptide (1): Xaa is a pyrrolidone carboxilic acid
XWFRIP

SEQ ID NO: 162 - protein, 6 aa, Bothrops jararaca
  mat_peptide (1): Xaa is a pyrrolidone carboxilic acid
XSFRIP

SEQ ID NO: 163 - protein, 6 aa, Bothrops jararaca
  mat_peptide (1): Xaa is a pyrrolidone carboxilic acid
XKFRIP

SEQ ID NO: 164 - protein, 6 aa, Bothrops jararaca
  mat_peptide (1): Xaa is a pyrrolidone carboxilic acid
XWGRIP

SEQ ID NO: 165 - protein, 6 aa, Bothrops jararaca
  mat_peptide (1): Xaa is a pyrrolidone carboxilic acid
XSGRIP

SEQ ID NO: 166 - protein, 6 aa, Bothrops jararaca
  mat_peptide (1): Xaa is a pyrrolidone carboxilic acid
XKGRIP

SEQ ID NO: 167 - protein, 6 aa, Bothrops jararaca
  mat_peptide (1): Xaa is a pyrrolidone carboxilic acid
XSWPGP

SEQ ID NO: 168 - protein, 6 aa, Bothrops jararaca
  mat_peptide (1): Xaa is a pyrrolidone carboxilic acid
XKWPGP

SEQ ID NO: 169 - protein, 6 aa, Bothrops jararaca
  mat_peptide (1): Xaa is a pyrrolidone carboxilic acid
XSPPGP

SEQ ID NO: 170 - protein, 6 aa, Bothrops jararaca
  mat_peptide (1): Xaa is a pyrrolidone carboxilic acid
XWFPGP

SEQ ID NO: 171 - protein, 6 aa, Bothrops jararaca
  mat_peptide (1): Xaa is a pyrrolidone carboxilic acid
XSFPGP

SEQ ID NO: 172 - protein, 6 aa, Bothrops jararaca
  mat_peptide (1): Xaa is a pyrrolidone carboxilic acid
XKFPGP

SEQ ID NO: 173 - protein, 6 aa, Bothrops jararaca
  mat_peptide (1): Xaa is a pyrrolidone carboxilic acid
XWGPGP

SEQ ID NO: 174 - protein, 6 aa, Bothrops jararaca
  mat_peptide (1): Xaa is a pyrrolidone carboxilic acid
XSGPGP

SEQ ID NO: 175 - protein, 6 aa, Bothrops jararaca
  mat_peptide (1): Xaa is a pyrrolidone carboxilic acid
XKGPGP

SEQ ID NO: 176 - protein, 6 aa, Bothrops jararaca
  mat_peptide (1): Xaa is a pyrrolidone carboxilic acid
XSWAGP

SEQ ID NO: 177 - protein, 6 aa, Bothrops jararaca
  mat_peptide (1): Xaa is a pyrrolidone carboxilic acid
XKWAGP

SEQ ID NO: 178 - protein, 6 aa, Bothrops jararaca
  mat_peptide (1): Xaa is a pyrrolidone carboxilic acid
XWPAGP

SEQ ID NO: 179 - protein, 6 aa, Bothrops jararaca
  mat_peptide (1): Xaa is a pyrrolidone carboxilic acid
XSPAGP

SEQ ID NO: 180 - protein, 6 aa, Bothrops jararaca
  mat_peptide (1): Xaa is a pyrrolidone carboxilic acid
XKPAGP

SEQ ID NO: 181 - protein, 6 aa, Bothrops jararaca
  mat_peptide (1): Xaa is a pyrrolidone carboxilic acid
XWFAGP

SEQ ID NO: 182 - protein, 6 aa, Bothrops jararaca
  mat_peptide (1): Xaa is a pyrrolidone carboxilic acid
XSFAGP

SEQ ID NO: 183 - protein, 6 aa, Bothrops jararaca
  mat_peptide (1): Xaa is a pyrrolidone carboxilic acid
XKFAGP

SEQ ID NO: 184 - protein, 6 aa, Bothrops jararaca
  mat_peptide (1): Xaa is a pyrrolidone carboxilic acid
XSWRGP

SEQ ID NO: 185 - protein, 6 aa, Bothrops jararaca
  mat_peptide (1): Xaa is a pyrrolidone carboxilic acid
XKWRGP

SEQ ID NO: 186 - protein, 6 aa, Bothrops jararaca
  mat_peptide (1): Xaa is a pyrrolidone carboxilic acid
XWPRGP

SEQ ID NO: 187 - protein, 6 aa, Bothrops jararaca
  mat_peptide (1): Xaa is a pyrrolidone carboxilic acid
XSPRGP

SEQ ID NO: 188 - protein, 6 aa, Bothrops jararaca
  mat_peptide (1): Xaa is a pyrrolidone carboxilic acid
XKPRGP

SEQ ID NO: 189 - protein, 6 aa, Bothrops jararaca
  mat_peptide (1): Xaa is a pyrrolidone carboxilic acid
XWFRGP

SEQ ID NO: 190 - protein, 6 aa, Bothrops jararaca
  mat_peptide (1): Xaa is a pyrrolidone carboxilic acid
XSFRGP

SEQ ID NO: 191 - protein, 6 aa, Bothrops jararaca
  mat_peptide (1): Xaa is a pyrrolidone carboxilic acid
XKFRGP

SEQ ID NO: 192 - protein, 6 aa, Bothrops jararaca
  mat_peptide (1): Xaa is a pyrrolidone carboxilic acid
XSWIGP

SEQ ID NO: 193 - protein, 6 aa, Bothrops jararaca
  mat_peptide (1): Xaa is a pyrrolidone carboxilic acid
XKWIGP

SEQ ID NO: 194 - protein, 6 aa, Bothrops jararaca
  mat_peptide (1): Xaa is a pyrrolidone carboxilic acid
XWPIGP

SEQ ID NO: 195 - protein, 6 aa, Bothrops jararaca
  mat_peptide (1): Xaa is a pyrrolidone carboxilic acid
XSPIGP

SEQ ID NO: 196 - protein, 6 aa, Bothrops jararaca
  mat_peptide (1): Xaa is a pyrrolidone carboxilic acid
XKPIGP

SEQ ID NO: 197 - protein, 6 aa, Bothrops jararaca
  mat_peptide (1): Xaa is a pyrrolidone carboxilic acid
XWFIGP

SEQ ID NO: 198 - protein, 6 aa, Bothrops jararaca
  mat_peptide (1): Xaa is a pyrrolidone carboxilic acid
XSFIGP

SEQ ID NO: 199 - protein, 6 aa, Bothrops jararaca
  mat_peptide (1): Xaa is a pyrrolidone carboxilic acid
XKFIGP

SEQ ID NO: 200 - protein, 6 aa, Bothrops jararaca
  mat_peptide (1): Xaa is a pyrrolidone carboxilic acid
XSWPHP

SEQ ID NO: 201 - protein, 6 aa, Bothrops jararaca
  mat_peptide (1): Xaa is a pyrrolidone carboxilic acid
XKWPHP

SEQ ID NO: 202 - protein, 6 aa, Bothrops jararaca
  mat_peptide (1): Xaa is a pyrrolidone carboxilic acid
XWPPHP

SEQ ID NO: 203 - protein, 6 aa, Bothrops jararaca
  mat_peptide (1): Xaa is a pyrrolidone carboxilic acid
XSPPHP

SEQ ID NO: 204 - protein, 6 aa, Bothrops jararaca
  mat_peptide (1): Xaa is a pyrrolidone carboxilic acid
XKPPHP

SEQ ID NO: 205 - protein, 6 aa, Bothrops jararaca
  mat_peptide (1): Xaa is a pyrrolidone carboxilic acid
XWFPHP

SEQ ID NO: 206 - protein, 6 aa, Bothrops jararaca
  mat_peptide (1): Xaa is a pyrrolidone carboxilic acid
XSFPHP

SEQ ID NO: 207 - protein, 6 aa, Bothrops jararaca
  mat_peptide (1): Xaa is a pyrrolidone carboxilic acid
XKFPHP

SEQ ID NO: 208 - protein, 6 aa, Bothrops jararaca
  mat_peptide (1): Xaa is a pyrrolidone carboxilic acid
XWGPHP

SEQ ID NO: 209 - protein, 6 aa, Bothrops jararaca
  mat_peptide (1): Xaa is a pyrrolidone carboxilic acid
XSGPHP

SEQ ID NO: 210 - protein, 6 aa, Bothrops jararaca
  mat_peptide (1): Xaa is a pyrrolidone carboxilic acid
XKGPHP

SEQ ID NO: 211 - protein, 6 aa, Bothrops jararaca
  mat_peptide (1): Xaa is a pyrrolidone carboxilic acid
XSWAHP

SEQ ID NO: 212 - protein, 6 aa, Bothrops jararaca
  mat_peptide (1): Xaa is a pyrrolidone carboxilic acid
XKWAHP

SEQ ID NO: 213 - protein, 6 aa, Bothrops jararaca
  mat_peptide (1): Xaa is a pyrrolidone carboxilic acid
XWPAHP

SEQ ID NO: 214 - protein, 6 aa, Bothrops jararaca
  mat_peptide (1): Xaa is a pyrrolidone carboxilic acid
XSPAHP

SEQ ID NO: 215 - protein, 6 aa, Bothrops jararaca
  mat_peptide (1): Xaa is a pyrrolidone carboxilic acid
XKPAHP

SEQ ID NO: 216 - protein, 6 aa, Bothrops jararaca
  mat_peptide (1): Xaa is a pyrrolidone carboxilic acid
XWFAHP

SEQ ID NO: 217 - protein, 6 aa, Bothrops jararaca
  mat_peptide (1): Xaa is a pyrrolidone carboxilic acid
XSFAHP

SEQ ID NO: 218 - protein, 6 aa, Bothrops jararaca
  mat_peptide (1): Xaa is a pyrrolidone carboxilic acid
XKFAHP

SEQ ID NO: 219 - protein, 6 aa, Bothrops jararaca
  mat_peptide (1): Xaa is a pyrrolidone carboxilic acid
XWGAHP

SEQ ID NO: 220 - protein, 6 aa, Bothrops jararaca
  mat_peptide (1): Xaa is a pyrrolidone carboxilic acid
XSGAHP

SEQ ID NO: 221 - protein, 6 aa, Bothrops jararaca
  mat_peptide (1): Xaa is a pyrrolidone carboxilic acid
XKGAHP

SEQ ID NO: 222 - protein, 6 aa, Bothrops jararaca
  mat_peptide (1): Xaa is a pyrrolidone carboxilic acid
XSWRHP

SEQ ID NO: 223 - protein, 6 aa, Bothrops jararaca
  mat_peptide (1): Xaa is a pyrrolidone carboxilic acid
XKWRHP

SEQ ID NO: 224 - protein, 6 aa, Bothrops jararaca
  mat_peptide (1): Xaa is a pyrrolidone carboxilic acid
XWPRHP

SEQ ID NO: 225 - protein, 6 aa, Bothrops jararaca
  mat_peptide (1): Xaa is a pyrrolidone carboxilic acid
XSPRHP

SEQ ID NO: 226 - protein, 6 aa, Bothrops jararaca
  mat_peptide (1): Xaa is a pyrrolidone carboxilic acid
XKPRHP

SEQ ID NO: 227 - protein, 6 aa, Bothrops jararaca
  mat_peptide (1): Xaa is a pyrrolidone carboxilic acid
XWFRHP

SEQ ID NO: 228 - protein, 6 aa, Bothrops jararaca
  mat_peptide (1): Xaa is a pyrrolidone carboxilic acid
XSFRHP

SEQ ID NO: 229 - protein, 6 aa, Bothrops jararaca
  mat_peptide (1): Xaa is a pyrrolidone carboxilic acid
XKFRHP

SEQ ID NO: 230 - protein, 6 aa, Bothrops jararaca
  mat_peptide (1): Xaa is a pyrrolidone carboxilic acid
XWGRHP

SEQ ID NO: 231 - protein, 6 aa, Bothrops jararaca
  mat_peptide (1): Xaa is a pyrrolidone carboxilic acid
XSGRHP

SEQ ID NO: 232 - protein, 6 aa, Bothrops jararaca
  mat_peptide (1): Xaa is a pyrrolidone carboxilic acid
XKGRHP

SEQ ID NO: 233 - protein, 6 aa, Bothrops jararaca
  mat_peptide (1): Xaa is a pyrrolidone carboxilic acid
XSWIHP

SEQ ID NO: 234 - protein, 6 aa, Bothrops jararaca
  mat_peptide (1): Xaa is a pyrrolidone carboxilic acid
XKWIHP

SEQ ID NO: 235 - protein, 6 aa, Bothrops jararaca
  mat_peptide (1): Xaa is a pyrrolidone carboxilic acid
XWPIHP

SEQ ID NO: 236 - protein, 6 aa, Bothrops jararaca
  mat_peptide (1): Xaa is a pyrrolidone carboxilic acid
XSPIHP

SEQ ID NO: 237 - protein, 6 aa, Bothrops jararaca
  mat_peptide (1): Xaa is a pyrrolidone carboxilic acid
XKPIHP

SEQ ID NO: 238 - protein, 6 aa, Bothrops jararaca
  mat_peptide (1): Xaa is a pyrrolidone carboxilic acid
XWFIHP

SEQ ID NO: 239 - protein, 6 aa, Bothrops jararaca
  mat_peptide (1): Xaa is a pyrrolidone carboxilic acid
XSFIHP

SEQ ID NO: 240 - protein, 6 aa, Bothrops jararaca
  mat_peptide (1): Xaa is a pyrrolidone carboxilic acid
XKFIHP

SEQ ID NO: 241 - protein, 6 aa, Bothrops jararaca
  mat_peptide (1): Xaa is a pyrrolidone carboxilic acid
XWGIHP

SEQ ID NO: 242 - protein, 6 aa, Bothrops jararaca
  mat_peptide (1): Xaa is a pyrrolidone carboxilic acid
XSGIHP

SEQ ID NO: 243 - protein, 6 aa, Bothrops jararaca
  mat_peptide (1): Xaa is a pyrrolidone carboxilic acid
XKGIHP

SEQ ID NO: 244 - protein, 7 aa, Bothrops jararaca
  mat_peptide (1): Xaa is a pyrrolidone carboxilic acid
XSWPRPP

SEQ ID NO: 245 - protein, 7 aa, Bothrops jararaca
  mat_peptide (1): Xaa is a pyrrolidone carboxilic acid
XSFPRPP

SEQ ID NO: 246 - protein, 7 aa, Bothrops jararaca
  mat_peptide (1): Xaa is a pyrrolidone carboxilic acid
XDGPRPP

SEQ ID NO: 247 - protein, 7 aa, Bothrops jararaca
  mat_peptide (1): Xaa is a pyrrolidone carboxilic acid
XWGPRPP

SEQ ID NO: 248 - protein, 7 aa, Bothrops jararaca
  mat_peptide (1): Xaa is a pyrrolidone carboxilic acid
XSGPRPP

SEQ ID NO: 249 - protein, 7 aa, Bothrops jararaca
  mat_peptide (1): Xaa is a pyrrolidone carboxilic acid
XDWPIPP

SEQ ID NO: 250 - protein, 7 aa, Bothrops jararaca
  mat_peptide (1): Xaa is a pyrrolidone carboxilic acid
XSWPIPP

SEQ ID NO: 251 - protein, 7 aa, Bothrops jararaca
  mat_peptide (1): Xaa is a pyrrolidone carboxilic acid
XWFPIPP

SEQ ID NO: 252 - protein, 7 aa, Bothrops jararaca
  mat_peptide (1): Xaa is a pyrrolidone carboxilic acid
XSFPIPP

SEQ ID NO: 253 - protein, 7 aa, Bothrops jararaca
  mat_peptide (1): Xaa is a pyrrolidone carboxilic acid
XDGPIPP

SEQ ID NO: 254 - protein, 7 aa, Bothrops jararaca
  mat_peptide (1): Xaa is a pyrrolidone carboxilic acid
XWGPIPP

SEQ ID NO: 255 - protein, 7 aa, Bothrops jararaca
  mat_peptide (1): Xaa is a pyrrolidone carboxilic acid
XSGPIPP

SEQ ID NO: 256 - protein, 7 aa, Bothrops jararaca
  mat_peptide (1): Xaa is a pyrrolidone carboxilic acid
XDWAIPP

SEQ ID NO: 257 - protein, 7 aa, Bothrops jararaca
  mat_peptide (1): Xaa is a pyrrolidone carboxilic acid
XSWAIPP

SEQ ID NO: 258 - protein, 7 aa, Bothrops jararaca
  mat_peptide (1): Xaa is a pyrrolidone carboxilic acid
XDPAIPP

SEQ ID NO: 259 - protein, 7 aa, Bothrops jararaca
  mat_peptide (1): Xaa is a pyrrolidone carboxilic acid
XWPAIPP

SEQ ID NO: 260 - protein, 7 aa, Bothrops jararaca
  mat_peptide (1): Xaa is a pyrrolidone carboxilic acid
XSPAIPP

SEQ ID NO: 261 - protein, 7 aa, Bothrops jararaca
  mat_peptide (1): Xaa is a pyrrolidone carboxilic acid
XDFAIPP

SEQ ID NO: 262 - protein, 7 aa, Bothrops jararaca
  mat_peptide (1): Xaa is a pyrrolidone carboxilic acid
XWFAIPP

SEQ ID NO: 263 - protein, 7 aa, Bothrops jararaca
  mat_peptide (1): Xaa is a pyrrolidone carboxilic acid
XSFAIPP

SEQ ID NO: 264 - protein, 7 aa, Bothrops jararaca
  mat_peptide (1): Xaa is a pyrrolidone carboxilic acid
XDGAIPP

SEQ ID NO: 265 - protein, 7 aa, Bothrops jararaca
  mat_peptide (1): Xaa is a pyrrolidone carboxilic acid
XWGAIPP

SEQ ID NO: 266 - protein, 7 aa, Bothrops jararaca
  mat_peptide (1): Xaa is a pyrrolidone carboxilic acid
XSGAIPP

SEQ ID NO: 267 - protein, 7 aa, Bothrops jararaca
  mat_peptide (1): Xaa is a pyrrolidone carboxilic acid
XDWRIPP

SEQ ID NO: 268 - protein, 7 aa, Bothrops jararaca
  mat_peptide (1): Xaa is a pyrrolidone carboxilic acid
XSWRIPP

SEQ ID NO: 269 - protein, 7 aa, Bothrops jararaca
  mat_peptide (1): Xaa is a pyrrolidone carboxilic acid
XDPRIPP

SEQ ID NO: 270 - protein, 7 aa, Bothrops jararaca
  mat_peptide (1): Xaa is a pyrrolidone carboxilic acid
XWPRIPP

SEQ ID NO: 271 - protein, 7 aa, Bothrops jararaca
  mat_peptide (1): Xaa is a pyrrolidone carboxilic acid
XSPRIPP

SEQ ID NO: 272 - protein, 7 aa, Bothrops jararaca
  mat_peptide (1): Xaa is a pyrrolidone carboxilic acid
XDFRIPP

SEQ ID NO: 273 - protein, 7 aa, Bothrops jararaca
  mat_peptide (1): Xaa is a pyrrolidone carboxilic acid
XWFRIPP

SEQ ID NO: 274 - protein, 7 aa, Bothrops jararaca
  mat_peptide (1): Xaa is a pyrrolidone carboxilic acid
XPFRIPP

SEQ ID NO: 275 - protein, 7 aa, Bothrops jararaca
  mat_peptide (1): Xaa is a pyrrolidone carboxilic acid
XDGRIPP

SEQ ID NO: 276 - protein, 7 aa, Bothrops jararaca
  mat_peptide (1): Xaa is a pyrrolidone carboxilic acid
XWGRIPP

SEQ ID NO: 277 - protein, 7 aa, Bothrops jararaca
  mat_peptide (1): Xaa is a pyrrolidone carboxilic acid
XSGRIPP

SEQ ID NO: 278 - protein, 7 aa, Bothrops jararaca
  mat_peptide (1): Xaa is a pyrrolidone carboxilic acid
XDWPRHP

SEQ ID NO: 279 - protein, 7 aa, Bothrops jararaca
  mat_peptide (1): Xaa is a pyrrolidone carboxilic acid
XSWPRHP

SEQ ID NO: 280 - protein, 7 aa, Bothrops jararaca
  mat_peptide (1): Xaa is a pyrrolidone carboxilic acid
XDPPRHP

SEQ ID NO: 281 - protein, 7 aa, Bothrops jararaca
  mat_peptide (1): Xaa is a pyrrolidone carboxilic acid
XWPPRHP

SEQ ID NO: 282 - protein, 7 aa, Bothrops jararaca
  mat_peptide (1): Xaa is a pyrrolidone carboxilic acid
XSPPRHP

SEQ ID NO: 283 - protein, 7 aa, Bothrops jararaca
  mat_peptide (1): Xaa is a pyrrolidone carboxilic acid
XDFPRHP

SEQ ID NO: 284 - protein, 7 aa, Bothrops jararaca
  mat_peptide (1): Xaa is a pyrrolidone carboxilic acid
XWFPRHP

SEQ ID NO: 285 - protein, 7 aa, Bothrops jararaca
  mat_peptide (1): Xaa is a pyrrolidone carboxilic acid
XSFPRHP

SEQ ID NO: 286 - protein, 7 aa, Bothrops jararaca
  mat_peptide (1): Xaa is a pyrrolidone carboxilic acid
XDGPRHP

SEQ ID NO: 287 - protein, 7 aa, Bothrops jararaca
  mat_peptide (1): Xaa is a pyrrolidone carboxilic acid
XWGPRHP

SEQ ID NO: 288 - protein, 7 aa, Bothrops jararaca
  mat_peptide (1): Xaa is a pyrrolidone carboxilic acid
XSGPRHP

SEQ ID NO: 289 - protein, 7 aa, Bothrops jararaca
  mat_peptide (1): Xaa is a pyrrolidone carboxilic acid
XDWPIHP

SEQ ID NO: 290 - protein, 7 aa, Bothrops jararaca
  mat_peptide (1): Xaa is a pyrrolidone carboxilic acid
XSWPIHP

SEQ ID NO: 291 - protein, 7 aa, Bothrops jararaca
  mat_peptide (1): Xaa is a pyrrolidone carboxilic acid
XDPPIHP

SEQ ID NO: 292 - protein, 7 aa, Bothrops jararaca
  mat_peptide (1): Xaa is a pyrrolidone carboxilic acid
XWPPIHP

SEQ ID NO: 293 - protein, 7 aa, Bothrops jararaca
  mat_peptide (1): Xaa is a pyrrolidone carboxilic acid
XSPPIHP

SEQ ID NO: 294 - protein, 7 aa, Bothrops jararaca
  mat_peptide (1): Xaa is a pyrrolidone carboxilic acid
XDFPIHP

SEQ ID NO: 295 - protein, 7 aa, Bothrops jararaca
  mat_peptide (1): Xaa is a pyrrolidone carboxilic acid
XWFPIHP

SEQ ID NO: 296 - protein, 7 aa, Bothrops jararaca
  mat_peptide (1): Xaa is a pyrrolidone carboxilic acid
XSFPIHP

SEQ ID NO: 297 - protein, 7 aa, Bothrops jararaca
  mat_peptide (1): Xaa is a pyrrolidone carboxilic acid
XDGPIHP

SEQ ID NO: 298 - protein, 7 aa, Bothrops jararaca
  mat_peptide (1): Xaa is a pyrrolidone carboxilic acid
XWGPIHP

SEQ ID NO: 299 - protein, 7 aa, Bothrops jararaca
  mat_peptide (1): Xaa is a pyrrolidone carboxilic acid
XSGPIHP

SEQ ID NO: 300 - protein, 7 aa, Bothrops jararaca
  mat_peptide (1): Xaa is a pyrrolidone carboxilic acid
XDWRIHP

SEQ ID NO: 301 - protein, 7 aa, Bothrops jararaca
  mat_peptide (1): Xaa is a pyrrolidone carboxilic acid
XSWRIHP

SEQ ID NO: 302 - protein, 7 aa, Bothrops jararaca
  mat_peptide (1): Xaa is a pyrrolidone carboxilic acid
XDPRIHP

SEQ ID NO: 303 - protein, 7 aa, Bothrops jararaca
  mat_peptide (1): Xaa is a pyrrolidone carboxilic acid
XWPRIHP

SEQ ID NO: 304 - protein, 7 aa, Bothrops jararaca
  mat_peptide (1): Xaa is a pyrrolidone carboxilic acid
XSPRIHP

SEQ ID NO: 305 - protein, 7 aa, Bothrops jararaca
  mat_peptide (1): Xaa is a pyrrolidone carboxilic acid
XDFRIHP

SEQ ID NO: 306 - protein, 7 aa, Bothrops jararaca
  mat_peptide (1): Xaa is a pyrrolidone carboxilic acid
XWFRIHP

SEQ ID NO: 307 - protein, 7 aa, Bothrops jararaca
  mat_peptide (1): Xaa is a pyrrolidone carboxilic acid
XSFRIHP

SEQ ID NO: 308 - protein, 7 aa, Bothrops jararaca
  mat_peptide (1): Xaa is a pyrrolidone carboxilic acid
XDGRIHP

SEQ ID NO: 309 - protein, 7 aa, Bothrops jararaca
  mat_peptide (1): Xaa is a pyrrolidone carboxilic acid
XWGRIHP

SEQ ID NO: 310 - protein, 7 aa, Bothrops jararaca
  mat_peptide (1): Xaa is a pyrrolidone carboxilic acid
XSGRIHP

SEQ ID NO: 311 - protein, 7 aa, Bothrops jararaca
  mat_peptide (1): Xaa is a pyrrolidone carboxilic acid
XSWPGPP

SEQ ID NO: 312 - protein, 7 aa, Bothrops jararaca
  mat_peptide (1): Xaa is a pyrrolidone carboxilic acid
XNWPGPP

SEQ ID NO: 313 - protein, 7 aa, Bothrops jararaca
  mat_peptide (1): Xaa is a pyrrolidone carboxilic acid
XWRPGPP

SEQ ID NO: 314 - protein, 7 aa, Bothrops jararaca
  mat_peptide (1): Xaa is a pyrrolidone carboxilic acid
XSRPGPP

SEQ ID NO: 315 - protein, 7 aa, Bothrops jararaca
  mat_peptide (1): Xaa is a pyrrolidone carboxilic acid
XNRPGPP

SEQ ID NO: 316 - protein, 7 aa, Bothrops jararaca
  mat_peptide (1): Xaa is a pyrrolidone carboxilic acid
XWGPZPP

SEQ ID NO: 317 - protein, 7 aa, Bothrops jararaca
  mat_peptide (1): Xaa is a pyrrolidone carboxilic acid
XSGPZPP

SEQ ID NO: 318 - protein, 7 aa, Bothrops jararaca
  mat_peptide (1): Xaa is a pyrrolidone carboxilic acid
XNGPZPP

SEQ ID NO: 319 - protein, 7 aa, Bothrops jararaca
  mat_peptide (1): Xaa is a pyrrolidone carboxilic acid
XSWPZPP

SEQ ID NO: 320 - protein, 7 aa, Bothrops jararaca
  mat_peptide (1): Xaa is a pyrrolidone carboxilic acid
XGWPZPP

SEQ ID NO: 321 - protein, 7 aa, Bothrops jararaca
  mat_peptide (1): Xaa is a pyrrolidone carboxilic acid
XNWPZPP

SEQ ID NO: 322 - protein, 7 aa, Bothrops jararaca
  mat_peptide (1): Xaa is a pyrrolidone carboxilic acid
XWRPZPP

SEQ ID NO: 323 - protein, 7 aa, Bothrops jararaca
  mat_peptide (1): Xaa is a pyrrolidone carboxilic acid
XSRPZPP

SEQ ID NO: 324 - protein, 7 aa, Bothrops jararaca
  mat_peptide (1): Xaa is a pyrrolidone carboxilic acid
XGRPZPP

SEQ ID NO: 325 - protein, 7 aa, Bothrops jararaca
  mat_peptide (1): Xaa is a pyrrolidone carboxilic acid
XNRPZPP

SEQ ID NO: 326 - protein, 9 aa, Bothrops jararaca
  mat_peptide (1): Xaa is a pyrrolidone carboxilic acid
XWPRPZIPP

SEQ ID NO: 327 - protein, 9 aa, Bothrops jararaca
  mat_peptide (1): Xaa is a pyrrolidone carboxilic acid
XSPRPZIPP

SEQ ID NO: 328 - protein, 9 aa, Bothrops jararaca
  mat_peptide (1): Xaa is a pyrrolidone carboxilic acid
XNPRPZIPP

SEQ ID NO: 329 - protein, 9 aa, Bothrops jararaca
  mat_peptide (1): Xaa is a pyrrolidone carboxilic acid
XSWRPZIPP

SEQ ID NO: 330 - protein, 9 aa, Bothrops jararaca
  mat_peptide (1): Xaa is a pyrrolidone carboxilic acid
XNWRPZIPP

SEQ ID NO: 331 - protein, 9 aa, Bothrops jararaca
  mat_peptide (1): Xaa is a pyrrolidone carboxilic acid
XWGRPZIPP

SEQ ID NO: 332 - protein, 9 aa, Bothrops jararaca
  mat_peptide (1): Xaa is a pyrrolidone carboxilic acid
XSGRPZIPP

SEQ ID NO: 333 - protein, 9 aa, Bothrops jararaca
  mat_peptide (1): Xaa is a pyrrolidone carboxilic acid
XNGRPZIPP

SEQ ID NO: 334 - protein, 9 aa, Bothrops jararaca
  mat_peptide (1): Xaa is a pyrrolidone carboxilic acid
XWPHPZIPP

SEQ ID NO: 335 - protein, 9 aa, Bothrops jararaca
  mat_peptide (1): Xaa is a pyrrolidone carboxilic acid
XSPHPZIPP

SEQ ID NO: 336 - protein, 9 aa, Bothrops jararaca
  mat_peptide (1): Xaa is a pyrrolidone carboxilic acid
XNPHPZIPP

SEQ ID NO: 337 - protein, 9 aa, Bothrops jararaca
  mat_peptide (1): Xaa is a pyrrolidone carboxilic acid
XSWHPZIPP

SEQ ID NO: 338 - protein, 9 aa, Bothrops jararaca
  mat_peptide (1): Xaa is a pyrrolidone carboxilic acid
XNWHPZIPP

SEQ ID NO: 339 - protein, 9 aa, Bothrops jararaca
  mat_peptide (1): Xaa is a pyrrolidone carboxilic acid
XWGHPZIPP

SEQ ID NO: 340 - protein, 9 aa, Bothrops jararaca
  mat_peptide (1): Xaa is a pyrrolidone carboxilic acid
XSGHPZIPP

SEQ ID NO: 341 - protein, 9 aa, Bothrops jararaca
  mat_peptide (1): Xaa is a pyrrolidone carboxilic acid
XNGHPZIPP

SEQ ID NO: 342 - protein, 9 aa, Bothrops jararaca
  mat_peptide (1): Xaa is a pyrrolidone carboxilic acid
XSWPPZIPP

SEQ ID NO: 343 - protein, 9 aa, Bothrops jararaca
  mat_peptide (1): Xaa is a pyrrolidone carboxilic acid
XNWPPZIPP

SEQ ID NO: 344 - protein, 9 aa, Bothrops jararaca
  mat_peptide (1): Xaa is a pyrrolidone carboxilic acid
XWGPPZIPP

SEQ ID NO: 345 - protein, 9 aa, Bothrops jararaca
  mat_peptide (1): Xaa is a pyrrolidone carboxilic acid
XSGPPZIPP

SEQ ID NO: 346 - protein, 9 aa, Bothrops jararaca
  mat_peptide (1): Xaa is a pyrrolidone carboxilic acid
XNGPPZIPP

SEQ ID NO: 347 - protein, 9 aa, Bothrops jararaca
  mat_peptide (1): Xaa is a pyrrolidone carboxilic acid
XWPRGZIPP

SEQ ID NO: 348 - protein, 9 aa, Bothrops jararaca
  mat_peptide (1): Xaa is a pyrrolidone carboxilic acid
XSPRGZIPP

SEQ ID NO: 349 - protein, 9 aa, Bothrops jararaca
  mat_peptide (1): Xaa is a pyrrolidone carboxilic acid
XNPRGZIPP

SEQ ID NO: 350 - protein, 9 aa, Bothrops jararaca
  mat_peptide (1): Xaa is a pyrrolidone carboxilic acid
XSWRGZIPP

SEQ ID NO: 351 - protein, 9 aa, Bothrops jararaca
  mat_peptide (1): Xaa is a pyrrolidone carboxilic acid
XNWRGZIPP

SEQ ID NO: 352 - protein, 9 aa, Bothrops jararaca
  mat_peptide (1): Xaa is a pyrrolidone carboxilic acid
XWPHGZIPP

SEQ ID NO: 353 - protein, 9 aa, Bothrops jararaca
  mat_peptide (1): Xaa is a pyrrolidone carboxilic acid
XSPHGZIPP

SEQ ID NO: 354 - protein, 9 aa, Bothrops jararaca
  mat_peptide (1): Xaa is a pyrrolidone carboxilic acid
XNPHGZIPP

SEQ ID NO: 355 - protein, 9 aa, Bothrops jararaca
  mat_peptide (1): Xaa is a pyrrolidone carboxilic acid
XSWHGZIPP

SEQ ID NO: 356 - protein, 9 aa, Bothrops jararaca
  mat_peptide (1): Xaa is a pyrrolidone carboxilic acid
XNWHGZIPP

SEQ ID NO: 357 - protein, 9 aa, Bothrops jararaca
  mat_peptide (1): Xaa is a pyrrolidone carboxilic acid
XWPPGZIPP

SEQ ID NO: 358 - protein, 9 aa, Bothrops jararaca
  mat_peptide (1): Xaa is a pyrrolidone carboxilic acid
XSPPGZIPP

SEQ ID NO: 359 - protein, 9 aa, Bothrops jararaca
  mat_peptide (1): Xaa is a pyrrolidone carboxilic acid
XNPPGZIPP

SEQ ID NO: 360 - protein, 9 aa, Bothrops jararaca
  mat_peptide (1): Xaa is a pyrrolidone carboxilic acid
XSWPGZIPP

SEQ ID NO: 361 - protein, 9 aa, Bothrops jararaca
  mat_peptide (1): Xaa is a pyrrolidone carboxilic acid
XNWPGZIPP

SEQ ID NO: 362 - protein, 9 aa, Bothrops jararaca
  mat_peptide (1): Xaa is a pyrrolidone carboxilic acid
XWPRPNIPP

SEQ ID NO: 363 - protein, 9 aa, Bothrops jararaca
  mat_peptide (1): Xaa is a pyrrolidone carboxilic acid
XSPRPNIPP

SEQ ID NO: 364 - protein, 9 aa, Bothrops jararaca
  mat_peptide (1): Xaa is a pyrrolidone carboxilic acid
XNPRPNIPP

SEQ ID NO: 365 - protein, 9 aa, Bothrops jararaca
  mat_peptide (1): Xaa is a pyrrolidone carboxilic acid
XSWRPNIPP

SEQ ID NO: 366 - protein, 9 aa, Bothrops jararaca
  mat_peptide (1): Xaa is a pyrrolidone carboxilic acid
XNWRPNIPP

SEQ ID NO: 367 - protein, 9 aa, Bothrops jararaca
  mat_peptide (1): Xaa is a pyrrolidone carboxilic acid
XWGRPNIPP

SEQ ID NO: 368 - protein, 9 aa, Bothrops jararaca
  mat_peptide (1): Xaa is a pyrrolidone carboxilic acid
XSGRPNIPP

SEQ ID NO: 369 - protein, 9 aa, Bothrops jararaca
  mat_peptide (1): Xaa is a pyrrolidone carboxilic acid
XNGRPNIPP

SEQ ID NO: 370 - protein, 9 aa, Bothrops jararaca
  mat_peptide (1): Xaa is a pyrrolidone carboxilic acid
XWPHPNIPP

SEQ ID NO: 371 - protein, 9 aa, Bothrops jararaca
  mat_peptide (1): Xaa is a pyrrolidone carboxilic acid
XSPHPNIPP

SEQ ID NO: 372 - protein, 9 aa, Bothrops jararaca
  mat_peptide (1): Xaa is a pyrrolidone carboxilic acid
XNPHPNIPP

SEQ ID NO: 373 - protein, 9 aa, Bothrops jararaca
  mat_peptide (1): Xaa is a pyrrolidone carboxilic acid
XSWHPNIPP

SEQ ID NO: 374 - protein, 9 aa, Bothrops jararaca
  mat_peptide (1): Xaa is a pyrrolidone carboxilic acid
XNWHPNIPP

SEQ ID NO: 375 - protein, 9 aa, Bothrops jararaca
  mat_peptide (1): Xaa is a pyrrolidone carboxilic acid
XWGHPNIPP

SEQ ID NO: 376 - protein, 9 aa, Bothrops jararaca
  mat_peptide (1): Xaa is a pyrrolidone carboxilic acid
XSGHPNIPP

SEQ ID NO: 377 - protein, 9 aa, Bothrops jararaca
  mat_peptide (1): Xaa is a pyrrolidone carboxilic acidy I, A, T or Y;
XNGHPNIPP

SEQ ID NO: 378 - protein, 9 aa, Bothrops jararaca
  mat_peptide (1): Xaa is a pyrrolidone carboxilic acid
XSWPPNIPP

SEQ ID NO: 379 - protein, 9 aa, Bothrops jararaca
  mat_peptide (1): Xaa is a pyrrolidone carboxilic acid
XNWPPNIPP

SEQ ID NO: 380 - protein, 9 aa, Bothrops jararaca
  mat_peptide (1): Xaa is a pyrrolidone carboxilic acid
XWGPPNIPP

SEQ ID NO: 381 - protein, 9 aa, Bothrops jararaca
  mat_peptide (1): Xaa is a pyrrolidone carboxilic acid
XSGPPNIPP

SEQ ID NO: 382 - protein, 9 aa, Bothrops jararaca
  mat_peptide (1): Xaa is a pyrrolidone carboxilic acid
XNGPPNIPP

SEQ ID NO: 383 - protein, 9 aa, Bothrops jararaca
  mat_peptide (1): Xaa is a pyrrolidone carboxilic acid
XWPRPZIPX

SEQ ID NO: 384 - protein, 9 aa, Bothrops jararaca
  mat_peptide (1): Xaa is a pyrrolidone carboxilic acid
XSPRPZIPX

SEQ ID NO: 385 - protein, 9 aa, Bothrops jararaca
  mat_peptide (1): Xaa is a pyrrolidone carboxilic acid
XNPRPZIPX

SEQ ID NO: 386 - protein, 9 aa, Bothrops jararaca
  mat_peptide (1): Xaa is a pyrrolidone carboxilic acid
XSWRPZIPX

SEQ ID NO: 387 - protein, 9 aa, Bothrops jararaca
  mat_peptide (1): Xaa is a pyrrolidone carboxilic acid
XNWRPZIPX

SEQ ID NO: 388 - protein, 9 aa, Bothrops jararaca
  mat_peptide (1): Xaa is a pyrrolidone carboxilic acid
XWGRPZIPX

SEQ ID NO: 389 - protein, 9 aa, Bothrops jararaca
  mat_peptide (1): Xaa is a pyrrolidone carboxilic acid
XSGRPZIPX

SEQ ID NO: 390 - protein, 9 aa, Bothrops jararaca
  mat_peptide (1): Xaa is a pyrrolidone carboxilic acid
XNGRPZIPX

SEQ ID NO: 391 - protein, 9 aa, Bothrops jararaca
  mat_peptide (1): Xaa is a pyrrolidone carboxilic acid
XWPHPZIPX

SEQ ID NO: 392 - protein, 9 aa, Bothrops jararaca
  mat_peptide (1): Xaa is a pyrrolidone carboxilic acid
XSPHPZIPX

SEQ ID NO: 393 - protein, 9 aa, Bothrops jararaca
  mat_peptide (1): Xaa is a pyrrolidone carboxilic acid
XNPHPZIPX

SEQ ID NO: 394 - protein, 9 aa, Bothrops jararaca
  mat_peptide (1): Xaa is a pyrrolidone carboxilic acid
XSWHPZIPX

SEQ ID NO: 395 - protein, 9 aa, Bothrops jararaca
  mat_peptide (1): Xaa is a pyrrolidone carboxilic acid
XNWHPZIPX

SEQ ID NO: 396 - protein, 9 aa, Bothrops jararaca
  mat_peptide (1): Xaa is a pyrrolidone carboxilic acid
XWGHPZIPX

SEQ ID NO: 397 - protein, 9 aa, Bothrops jararaca
  mat_peptide (1): Xaa is a pyrrolidone carboxilic acid
XSGHPZIPX

SEQ ID NO: 398 - protein, 9 aa, Bothrops jararaca
  mat_peptide (1): Xaa is a pyrrolidone carboxilic acid
XNGHPZIPX

SEQ ID NO: 399 - protein, 9 aa, Bothrops jararaca
  mat_peptide (1): Xaa is a pyrrolidone carboxilic acid
XSWPPZIPX

SEQ ID NO: 400 - protein, 9 aa, Bothrops jararaca
  mat_peptide (1): Xaa is a pyrrolidone carboxilic acid
XWGPPZIPX

SEQ ID NO: 401 - protein, 9 aa, Bothrops jararaca
  mat_peptide (1): Xaa is a pyrrolidone carboxilic acid
XSGPPZIPX

SEQ ID NO: 402 - protein, 9 aa, Bothrops jararaca
  mat_peptide (1): Xaa is a pyrrolidone carboxilic acid
XNGPPZIPX

SEQ ID NO: 403 - protein, 9 aa, Bothrops jararaca
  mat_peptide (1): Xaa is a pyrrolidone carboxilic acid
XWPRPZIPX

SEQ ID NO: 404 - protein, 9 aa, Bothrops jararaca
  mat_peptide (1): Xaa is a pyrrolidone carboxilic acid
XSPRPZIPX

SEQ ID NO: 405 - protein, 9 aa, Bothrops jararaca
  mat_peptide (1): Xaa is a pyrrolidone carboxilic acid
XNPRPZIPX

SEQ ID NO: 406 - protein, 9 aa, Bothrops jararaca
  mat_peptide (1): Xaa is a pyrrolidone carboxilic acid
XSWRPZIPX

SEQ ID NO: 407 - protein, 9 aa, Bothrops jararaca
  mat_peptide (1): Xaa is a pyrrolidone carboxilic acid
XNWRPZIPX

SEQ ID NO: 408 - protein, 9 aa, Bothrops jararaca
  mat_peptide (1): Xaa is a pyrrolidone carboxilic acid
XWGRPZIPX

SEQ ID NO: 409 - protein, 9 aa, Bothrops jararaca
  mat_peptide (1): Xaa is a pyrrolidone carboxilic acid
XSGRPZIPX

SEQ ID NO: 410 - protein, 9 aa, Bothrops jararaca
  mat_peptide (1): Xaa is a pyrrolidone carboxilic acid
XNGRPZIPX

SEQ ID NO: 411 - protein, 9 aa, Bothrops jararaca
  mat_peptide (1): Xaa is a pyrrolidone carboxilic acid
XWPHPZIPX

SEQ ID NO: 412 - protein, 9 aa, Bothrops jararaca
  mat_peptide (1): Xaa is a pyrrolidone carboxilic acid
XSPHPZIPX

SEQ ID NO: 413 - protein, 9 aa, Bothrops jararaca
  mat_peptide (1): Xaa is a pyrrolidone carboxilic acid
XNPHPZIPX

SEQ ID NO: 414 - protein, 9 aa, Bothrops jararaca
  mat_peptide (1): Xaa is a pyrrolidone carboxilic acid
XSWHPZIPX

SEQ ID NO: 415 - protein, 9 aa, Bothrops jararaca
  mat_peptide (1): Xaa is a pyrrolidone carboxilic acid
XNWHPZIPX

SEQ ID NO: 416 - protein, 9 aa, Bothrops jararaca
  mat_peptide (1): Xaa is a pyrrolidone carboxilic acid
XWGHPZIPX

SEQ ID NO: 417 - protein, 9 aa, Bothrops jararaca
  mat_peptide (1): Xaa is a pyrrolidone carboxilic acid
XSGHPZIPX

SEQ ID NO: 418 - protein, 9 aa, Bothrops jararaca
  mat_peptide (1): Xaa is a pyrrolidone carboxilic acid
XNGHPZIPX

SEQ ID NO: 419 - protein, 9 aa, Bothrops jararaca
  mat_peptide (1): Xaa is a pyrrolidone carboxilic acid
XSWPPZIPX

SEQ ID NO: 420 - protein, 9 aa, Bothrops jararaca
  mat_peptide (1): Xaa is a pyrrolidone carboxilic acid
XWGPPZIPX

SEQ ID NO: 421 - protein, 9 aa, Bothrops jararaca
  mat_peptide (1): Xaa is a pyrrolidone carboxilic acid
XSGPPZIPX

SEQ ID NO: 422 - protein, 9 aa, Bothrops jararaca
  mat_peptide (1): Xaa is a pyrrolidone carboxilic acid
XNGPPZIPX

SEQ ID NO: 423 - protein, 10 aa, Bothrops jararaca
  mat_peptide (1): Xaa is a pyrrolidone carboxilic acid
XSWPGPNIPP

SEQ ID NO: 424 - protein, 10 aa, Bothrops jararaca
  mat_peptide (1): Xaa is a pyrrolidone carboxilic acid
XNWPGPNIPP

SEQ ID NO: 425 - protein, 10 aa, Bothrops jararaca
  mat_peptide (1): Xaa is a pyrrolidone carboxilic acid
XSFPGPNIPP

SEQ ID NO: 426 - protein, 10 aa, Bothrops jararaca
  mat_peptide (1): Xaa is a pyrrolidone carboxilic acid
XNFPGPNIPP

SEQ ID NO: 427 - protein, 10 aa, Bothrops jararaca
  mat_peptide (1): Xaa is a pyrrolidone carboxilic acid
XWFPGPNIPP

SEQ ID NO: 428 - protein, 10 aa, Bothrops jararaca
  mat_peptide (1): Xaa is a pyrrolidone carboxilic acid
XSPPGPNIPP

SEQ ID NO: 429 - protein, 10 aa, Bothrops jararaca
  mat_peptide (1): Xaa is a pyrrolidone carboxilic acid
XNPPGPNIPP

SEQ ID NO: 430 - protein, 10 aa, Bothrops jararaca
  mat_peptide (1): Xaa is a pyrrolidone carboxilic acid
XWPPGPNIPP

SEQ ID NO: 431 - protein, 10 aa, Bothrops jararaca
  mat_peptide (1): Xaa is a pyrrolidone carboxilic acid
XSWPRPNIPP

SEQ ID NO: 432 - protein, 10 aa, Bothrops jararaca
  mat_peptide (1): Xaa is a pyrrolidone carboxilic acid
XNWPRPNIPP

SEQ ID NO: 433 - protein, 10 aa, Bothrops jararaca
  mat_peptide (1): Xaa is a pyrrolidone carboxilic acid
XSFPRPNIPP

SEQ ID NO: 434 - protein, 10 aa, Bothrops jararaca
  mat_peptide (1): Xaa is a pyrrolidone carboxilic acid
XNFPRPNIPP

SEQ ID NO: 435 - protein, 10 aa, Bothrops jararaca
  mat_peptide (1): Xaa is a pyrrolidone carboxilic acid
XWFPRPNIPP

SEQ ID NO: 436 - protein, 10 aa, Bothrops jararaca
  mat_peptide (1): Xaa is a pyrrolidone carboxilic acid
XSPPRPNIPP

SEQ ID NO: 437 - protein, 10 aa, Bothrops jararaca
  mat_peptide (1): Xaa is a pyrrolidone carboxilic acid
XNPPRPNIPP

SEQ ID NO: 438 - protein, 10 aa, Bothrops jararaca
  mat_peptide (1): Xaa is a pyrrolidone carboxilic acid
XWPPRPNIPP

SEQ ID NO: 439 - protein, 10 aa, Bothrops jararaca
  mat_peptide (1): Xaa is a pyrrolidone carboxilic acid
XSWPHPNIPP

SEQ ID NO: 440 - protein, 10 aa, Bothrops jararaca
  mat_peptide (1): Xaa is a pyrrolidone carboxilic acid
XNWPHPNIPP

SEQ ID NO: 441 - protein, 10 aa, Bothrops jararaca
  mat_peptide (1): Xaa is a pyrrolidone carboxilic acid
XSFPHPNIPP

SEQ ID NO: 442 - protein, 10 aa, Bothrops jararaca
  mat_peptide (1): Xaa is a pyrrolidone carboxilic acid
XNFPHPNIPP

SEQ ID NO: 443 - protein, 10 aa, Bothrops jararaca
  mat_peptide (1): Xaa is a pyrrolidone carboxilic acid
XWFPHPNIPP

SEQ ID NO: 444 - protein, 10 aa, Bothrops jararaca
  mat_peptide (1): Xaa is a pyrrolidone carboxilic acid
XSPPHPNIPP

SEQ ID NO: 445 - protein, 10 aa, Bothrops jararaca
  mat_peptide (1): Xaa is a pyrrolidone carboxilic acid
XNPPHPNIPP

SEQ ID NO: 446 - protein, 10 aa, Bothrops jararaca
  mat_peptide (1): Xaa is a pyrrolidone carboxilic acid
XWPPHPNIPP

SEQ ID NO: 447 - protein, 10 aa, Bothrops jararaca
  mat_peptide (1): Xaa is a pyrrolidone carboxilic acid
XSWPZPNIPP

SEQ ID NO: 448 - protein, 10 aa, Bothrops jararaca
  mat_peptide (1): Xaa is a pyrrolidone carboxilic acid
XNWPZPNIPP

SEQ ID NO: 449 - protein, 10 aa, Bothrops jararaca
  mat_peptide (1): Xaa is a pyrrolidone carboxilic acid
XSFPZPNIPP

SEQ ID NO: 450 - protein, 10 aa, Bothrops jararaca
  mat_peptide (1): Xaa is a pyrrolidone carboxilic acid
XNFPZPNIPP

SEQ ID NO: 451 - protein, 10 aa, Bothrops jararaca
  mat_peptide (1): Xaa is a pyrrolidone carboxilic acid
XWFPZPNIPP

SEQ ID NO: 452 - protein, 10 aa, Bothrops jararaca
  mat_peptide (1): Xaa is a pyrrolidone carboxilic acid
XSPPZPNIPP

SEQ ID NO: 453 - protein, 10 aa, Bothrops jararaca
  mat_peptide (1): Xaa is a pyrrolidone carboxilic acid
XNPPZPNIPP

SEQ ID NO: 454 - protein, 10 aa, Bothrops jararaca
  mat_peptide (1): Xaa is a pyrrolidone carboxilic acid
XWPPZPNIPP

SEQ ID NO: 455 - protein, 10 aa, Bothrops jararaca
  mat_peptide (1): Xaa is a pyrrolidone carboxilic acid
XSWPGPZIPP

SEQ ID NO: 456 - protein, 10 aa, Bothrops jararaca
  mat_peptide (1): Xaa is a pyrrolidone carboxilic acid
XNWPGPZIPP

SEQ ID NO: 457 - protein, 10 aa, Bothrops jararaca
  mat_peptide (1): Xaa is a pyrrolidone carboxilic acid
XSFPGPZIPP

SEQ ID NO: 458 - protein, 10 aa, Bothrops jararaca
  mat_peptide (1): Xaa is a pyrrolidone carboxilic acid
XNFPGPZIPP

SEQ ID NO: 459 - protein, 10 aa, Bothrops jararaca
  mat_peptide (1): Xaa is a pyrrolidone carboxilic acid
XWFPGPZIPP

SEQ ID NO: 460 - protein, 10 aa, Bothrops jararaca
  mat_peptide (1): Xaa is a pyrrolidone carboxilic acid
XSPPGPZIPP

SEQ ID NO: 461 - protein, 10 aa, Bothrops jararaca
  mat_peptide (1): Xaa is a pyrrolidone carboxilic acid
XNPPGPZIPP

SEQ ID NO: 462 - protein, 10 aa, Bothrops jararaca
  mat_peptide (1): Xaa is a pyrrolidone carboxilic acid
XWPPGPZIPP

SEQ ID NO: 463 - protein, 10 aa, Bothrops jararaca
  mat_peptide (1): Xaa is a pyrrolidone carboxilic acid
XSWPRPZIPP

SEQ ID NO: 464 - protein, 10 aa, Bothrops jararaca
  mat_peptide (1): Xaa is a pyrrolidone carboxilic acid
XNWPRPZIPP

SEQ ID NO: 465 - protein, 10 aa, Bothrops jararaca
  mat_peptide (1): Xaa is a pyrrolidone carboxilic acid
XSFPRPZIPP

SEQ ID NO: 466 - protein, 10 aa, Bothrops jararaca
  mat_peptide (1): Xaa is a pyrrolidone carboxilic acid
XNFPRPZIPP

SEQ ID NO: 467 - protein, 10 aa, Bothrops jararaca
  mat_peptide (1): Xaa is a pyrrolidone carboxilic acid
XWFPRPZIPP

SEQ ID NO: 468 - protein, 10 aa, Bothrops jararaca
  mat_peptide (1): Xaa is a pyrrolidone carboxilic acid
XSPPRPZIPP

SEQ ID NO: 469 - protein, 10 aa, Bothrops jararaca
  mat_peptide (1): Xaa is a pyrrolidone carboxilic acid
XNPPRPZIPP

SEQ ID NO: 470 - protein, 10 aa, Bothrops jararaca
  mat_peptide (1): Xaa is a pyrrolidone carboxilic acid
XWPPRPZIPP

SEQ ID NO: 471 - protein, 10 aa, Bothrops jararaca
  mat_peptide (1): Xaa is a pyrrolidone carboxilic acid
XSWPHPZIPP

SEQ ID NO: 472 - protein, 10 aa, Bothrops jararaca
  mat_peptide (1): Xaa is a pyrrolidone carboxilic acid
XNWPHPZIPP

SEQ ID NO: 473 - protein, 10 aa, Bothrops jararaca
  mat_peptide (1): Xaa is a pyrrolidone carboxilic acid
XSFPHPZIPP

SEQ ID NO: 474 - protein, 10 aa, Bothrops jararaca
  mat_peptide (1): Xaa is a pyrrolidone carboxilic acid
XNFPHPZIPP

SEQ ID NO: 475 - protein, 10 aa, Bothrops jararaca
  mat_peptide (1): Xaa is a pyrrolidone carboxilic acid
XWFPHPZIPP

SEQ ID NO: 476 - protein, 10 aa, Bothrops jararaca
  mat_peptide (1): Xaa is a pyrrolidone carboxilic acid
XSPPHPZIPP

SEQ ID NO: 477 - protein, 10 aa, Bothrops jararaca
  mat_peptide (1): Xaa is a pyrrolidone carboxilic acid
XNPPHPZIPP

SEQ ID NO: 478 - protein, 10 aa, Bothrops jararaca
  mat_peptide (1): Xaa is a pyrrolidone carboxilic acid
XWPPHPZIPP

SEQ ID NO: 479 - protein, 10 aa, Bothrops jararaca
  mat_peptide (1): Xaa is a pyrrolidone carboxilic acid
XSWPZPZIPP

SEQ ID NO: 480 - protein, 10 aa, Bothrops jararaca
  mat_peptide (1): Xaa is a pyrrolidone carboxilic acid
XNWPZPZIPP

SEQ ID NO: 481 - protein, 10 aa, Bothrops jararaca
  mat_peptide (1): Xaa is a pyrrolidone carboxilic acid
XSFPZPZIPP

SEQ ID NO: 482 - protein, 10 aa, Bothrops jararaca
  mat_peptide (1): Xaa is a pyrrolidone carboxilic acid
XNFPZPZIPP

SEQ ID NO: 483 - protein, 10 aa, Bothrops jararaca
  mat_peptide (1): Xaa is a pyrrolidone carboxilic acid
XWFPZPZIPP

SEQ ID NO: 484 - protein, 10 aa, Bothrops jararaca
  mat_peptide (1): Xaa is a pyrrolidone carboxilic acid
XSPPZPZIPP

SEQ ID NO: 485 - protein, 10 aa, Bothrops jararaca
  mat_peptide (1): Xaa is a pyrrolidone carboxilic acid
XNPPZPZIPP

SEQ ID NO: 486 - protein, 10 aa, Bothrops jararaca
  mat_peptide (1): Xaa is a pyrrolidone carboxilic acid
XWPPZPZIPP

SEQ ID NO: 487 - protein, 10 aa, Bothrops jararaca
  mat_peptide (1): Xaa is a pyrrolidone carboxilic acid
XSWPGPNIPX

SEQ ID NO: 488 - protein, 10 aa, Bothrops jararaca
  mat_peptide (1): Xaa is a pyrrolidone carboxilic acid
XNWPGPNIPX

SEQ ID NO: 489 - protein, 10 aa, Bothrops jararaca
  mat_peptide (1): Xaa is a pyrrolidone carboxilic acid
XNWPRPNIPX

SEQ ID NO: 490 - protein, 10 aa, Bothrops jararaca
  mat_peptide (1): Xaa is a pyrrolidone carboxilic acid
XNWPHPNIPX

SEQ ID NO: 491 - protein, 10 aa, Bothrops jararaca
  mat_peptide (1): Xaa is a pyrrolidone carboxilic acid
XNFPHPNIPX

SEQ ID NO: 492 - protein, 10 aa, Bothrops jararaca
  mat_peptide (1): Xaa is a pyrrolidone carboxilic acid
XWFPHPNIPX

SEQ ID NO: 493 - protein, 10 aa, Bothrops jararaca
  mat_peptide (1): Xaa is a pyrrolidone carboxilic acid
XSPPHPNIPX

SEQ ID NO: 494 - protein, 10 aa, Bothrops jararaca
  mat_peptide (1): Xaa is a pyrrolidone carboxilic acid
XNWPZPNIPX

SEQ ID NO: 495 - protein, 10 aa, Bothrops jararaca
  mat_peptide (1): Xaa is a pyrrolidone carboxilic acid
XSFPZPNIPX

SEQ ID NO: 496 - protein, 10 aa, Bothrops jararaca
  mat_peptide (1): Xaa is a pyrrolidone carboxilic acid
XWFPZPNIPX

SEQ ID NO: 497 - protein, 10 aa, Bothrops jararaca
  mat_peptide (1): Xaa is a pyrrolidone carboxilic acid
XSWPRPZIPX

SEQ ID NO: 498 - protein, 10 aa, Bothrops jararaca
  mat_peptide (1): Xaa is a pyrrolidone carboxilic acid
XNWPRPZIPX

SEQ ID NO: 499 - protein, 10 aa, Bothrops jararaca
  mat_peptide (1): Xaa is a pyrrolidone carboxilic acid
XNWPHPZIPX

SEQ ID NO: 500 - protein, 10 aa, Bothrops jararaca
  mat_peptide (1): Xaa is a pyrrolidone carboxilic acid
XSWPGPNIPX

SEQ ID NO: 501 - protein, 10 aa, Bothrops jararaca
  mat_peptide (1): Xaa is a pyrrolidone carboxilic acid
XNWPGPNIPX

SEQ ID NO: 502 - protein, 10 aa, Bothrops jararaca
  mat_peptide (1): Xaa is a pyrrolidone carboxilic acid
XNWPRPNIPX

SEQ ID NO: 503 - protein, 10 aa, Bothrops jararaca
  mat_peptide (1): Xaa is a pyrrolidone carboxilic acid
XNWPHPNIPX

SEQ ID NO: 504 - protein, 10 aa, Bothrops jararaca
  mat_peptide (1): Xaa is a pyrrolidone carboxilic acid
XNFPHPNIPX

SEQ ID NO: 505 - protein, 10 aa, Bothrops jararaca
  mat_peptide (1): Xaa is a pyrrolidone carboxilic acid
XWFPHPNIPX

SEQ ID NO: 506 - protein, 10 aa, Bothrops jararaca
  mat_peptide (1): Xaa is a pyrrolidone carboxilic acid
XSPPHPNIPX

SEQ ID NO: 507 - protein, 10 aa, Bothrops jararaca
  mat_peptide (1): Xaa is a pyrrolidone carboxilic acid
XNWPZPNIPX

SEQ ID NO: 508 - protein, 10 aa, Bothrops jararaca
  mat_peptide (1): Xaa is a pyrrolidone carboxilic acid
XSFPZPNIPX

SEQ ID NO: 509 - protein, 10 aa, Bothrops jararaca
  mat_peptide (1): Xaa is a pyrrolidone carboxilic acid
XWFPZPNIPX

SEQ ID NO: 510 - protein, 10 aa, Bothrops jararaca
  mat_peptide (1): Xaa is a pyrrolidone carboxilic acid
XSWPRPZIPX

SEQ ID NO: 511 - protein, 10 aa, Bothrops jararaca
  mat_peptide (1): Xaa is a pyrrolidone carboxilic acid
XNWPRPZIPX

SEQ ID NO: 512 - protein, 10 aa, Bothrops jararaca
  mat_peptide (1): Xaa is a pyrrolidone carboxilic acid
XNWPHPZIPX

SEQ ID NO: 513 - protein, 10 aa, Bothrops jararaca
  mat_peptide (1): Xaa is a pyrrolidone carboxilic acid
XWPPGPZIPP

SEQ ID NO: 514 - protein, 10 aa, Bothrops jararaca
  mat_peptide (1): Xaa is a pyrrolidone carboxilic acid
XWPPHPPIPP

SEQ ID NO: 515 - protein, 10 aa, Bothrops jararaca
  mat_peptide (1): Xaa is a pyrrolidone carboxilic acid
XWRPHPPIPP

SEQ ID NO: 516 - protein, 10 aa, Bothrops jararaca
  mat_peptide (1): Xaa is a pyrrolidone carboxilic acid
XGRPHPPIPP

SEQ ID NO: 517 - protein, 11 aa, Bothrops jararaca
  mat_peptide (1): Xaa is a pyrrolidone carboxilic acid
XWRPPTPZIPP

SEQ ID NO: 518 - protein, 11 aa, Bothrops jararaca
  mat_peptide (1): Xaa is a pyrrolidone carboxilic acid
XGRPPTPZIPP

SEQ ID NO: 519 - protein, 11 aa, Bothrops jararaca
  mat_peptide (1): Xaa is a pyrrolidone carboxilic acid
XARPPTPZIPP

SEQ ID NO: 520 - protein, 11 aa, Bothrops jararaca
  mat_peptide (1): Xaa is a pyrrolidone carboxilic acid
XWPAPTPZIPP

SEQ ID NO: 521 - protein, 11 aa, Bothrops jararaca
  mat_peptide (1): Xaa is a pyrrolidone carboxilic acid
XGPAPTPZIPP

SEQ ID NO: 522 - protein, 11 aa, Bothrops jararaca
  mat_peptide (1): Xaa is a pyrrolidone carboxilic acid
XAPAPTPZIPP

SEQ ID NO: 523 - protein, 11 aa, Bothrops jararaca
  mat_peptide (1): Xaa is a pyrrolidone carboxilic acid
XWRAPTPZIPP

SEQ ID NO: 524 - protein, 11 aa, Bothrops jararaca
  mat_peptide (1): Xaa is a pyrrolidone carboxilic acid
XGRAPTPZIPP

SEQ ID NO: 525 - protein, 11 aa, Bothrops jararaca
  mat_peptide (1): Xaa is a pyrrolidone carboxilic acid
XARAPTPZIPP

SEQ ID NO: 526 - protein, 11 aa, Bothrops jararaca
  mat_peptide (1): Xaa is a pyrrolidone carboxilic acid
XWPRPTPZIPP

SEQ ID NO: 527 - protein, 11 aa, Bothrops jararaca
  mat_peptide (1): Xaa is a pyrrolidone carboxilic acid
XGPRPTPZIPP

SEQ ID NO: 528 - protein, 11 aa, Bothrops jararaca
  mat_peptide (1): Xaa is a pyrrolidone carboxilic acid
XAPRPTPZIPP

SEQ ID NO: 529 - protein, 11 aa, Bothrops jararaca
  mat_peptide (1): Xaa is a pyrrolidone carboxilic acid
XWRPPGPZIPP

SEQ ID NO: 530 - protein, 11 aa, Bothrops jararaca
  mat_peptide (1): Xaa is a pyrrolidone carboxilic acid
XGRPPGPZIPP

SEQ ID NO: 531 - protein, 11 aa, Bothrops jararaca
  mat_peptide (1): Xaa is a pyrrolidone carboxilic acid
XARPPGPZIPP

SEQ ID NO: 532 - protein, 11 aa, Bothrops jararaca
  mat_peptide (1): Xaa is a pyrrolidone carboxilic acid
XWPAPGPZIPP

SEQ ID NO: 533 - protein, 11 aa, Bothrops jararaca
  mat_peptide (1): Xaa is a pyrrolidone carboxilic acid
XWPRPGPZIPP

SEQ ID NO: 534 - protein, 11 aa, Bothrops jararaca
  mat_peptide (1): Xaa is a pyrrolidone carboxilic acid
XGPRPGPZIPP

SEQ ID NO: 535 - protein, 11 aa, Bothrops jararaca
  mat_peptide (1): Xaa is a pyrrolidone carboxilic acid
XAPRPGPZIPP

SEQ ID NO: 536 - protein, 11 aa, Bothrops jararaca
  mat_peptide (1): Xaa is a pyrrolidone carboxilic acid
XGRPPHPZIPP

SEQ ID NO: 537 - protein, 11 aa, Bothrops jararaca
  mat_peptide (1): Xaa is a pyrrolidone carboxilic acid
XARPPHPZIPP

SEQ ID NO: 538 - protein, 11 aa, Bothrops jararaca
  mat_peptide (1): Xaa is a pyrrolidone carboxilic acid
XWPAPHPZIPP

SEQ ID NO: 539 - protein, 11 aa, Bothrops jararaca
  mat_peptide (1): Xaa is a pyrrolidone carboxilic acid
XWPRPHPZIPP

SEQ ID NO: 540 - protein, 11 aa, Bothrops jararaca
  mat_peptide (1): Xaa is a pyrrolidone carboxilic acid
XGPRPHPZIPP

SEQ ID NO: 541 - protein, 11 aa, Bothrops jararaca
  mat_peptide (1): Xaa is a pyrrolidone carboxilic acid
XAPRPHPZIPP

SEQ ID NO: 542 - protein, 11 aa, Bothrops jararaca
  mat_peptide (1): Xaa is a pyrrolidone carboxilic acid
XWRPPTPPIPP

SEQ ID NO: 543 - protein, 11 aa, Bothrops jararaca
  mat_peptide (1): Xaa is a pyrrolidone carboxilic acid
XGRPPTPPIPP

SEQ ID NO: 544 - protein, 11 aa, Bothrops jararaca
  mat_peptide (1): Xaa is a pyrrolidone carboxilic acid
XWPAPTPPIPP

SEQ ID NO: 545 - protein, 11 aa, Bothrops jararaca
  mat_peptide (1): Xaa is a pyrrolidone carboxilic acid
XWPRPTPPIPP

SEQ ID NO: 546 - protein, 11 aa, Bothrops jararaca
  mat_peptide (1): Xaa is a pyrrolidone carboxilic acid
XGPRPTPPIPP

SEQ ID NO: 547 - protein, 11 aa, Bothrops jararaca
  mat_peptide (1): Xaa is a pyrrolidone carboxilic acid
XAPRPTPPIPP

SEQ ID NO: 548 - protein, 11 aa, Bothrops jararaca
  mat_peptide (1): Xaa is a pyrrolidone carboxilic acid
XWRPPGPPIPP

SEQ ID NO: 549 - protein, 11 aa, Bothrops jararaca
  mat_peptide (1): Xaa is a pyrrolidone carboxilic acid
XWPAPGPPIPP

SEQ ID NO: 550 - protein, 11 aa, Bothrops jararaca
  mat_peptide (1): Xaa is a pyrrolidone carboxilic acid
XGPAPGPPIPP

SEQ ID NO: 551 - protein, 11 aa, Bothrops jararaca
  mat_peptide (1): Xaa is a pyrrolidone carboxilic acid
XWRPPHPPIPP

SEQ ID NO: 552 - protein, 11 aa, Bothrops jararaca
  mat_peptide (1): Xaa is a pyrrolidone carboxilic acid
XGRAPGPPIPP

SEQ ID NO: 553 - protein, 11 aa, Bothrops jararaca
  mat_peptide (1): Xaa is a pyrrolidone carboxilic acid
XGRAPHPPIPP

SEQ ID NO: 554 - protein, 11 aa, Bothrops jararaca
  mat_peptide (1): Xaa is a pyrrolidone carboxilic acid
XGRPPGPPIPP

SEQ ID NO: 555 - protein, 11 aa, Bothrops jararaca
  mat_peptide (1): Xaa is a pyrrolidone carboxilic acid
XARPPHPPIPP

SEQ ID NO: 556 - protein, 11 aa, Bothrops jararaca
  mat_peptide (1): Xaa is a pyrrolidone carboxilic acid
XWPRPTPQIPP

SEQ ID NO: 557 - protein, 12 aa, Bothrops jararaca
  mat_peptide (1): Xaa is a pyrrolidone carboxilic acid
XWGRPPGPPIPP

SEQ ID NO: 558 - protein, 12 aa, Bothrops jararaca
  mat_peptide (1): Xaa is a pyrrolidone carboxilic acid
XGWRPPGPPIPP

SEQ ID NO: 559 - protein, 12 aa, Bothrops jararaca
  mat_peptide (1): Xaa is a pyrrolidone carboxilic acid
XWGAPPGPPIPP

SEQ ID NO: 560 - protein, 12 aa, Bothrops jararaca
  mat_peptide (1): Xaa is a pyrrolidone carboxilic acid
XGWAPPGPPIPP

SEQ ID NO: 561 - protein, 12 aa, Bothrops jararaca
  mat_peptide (1): Xaa is a pyrrolidone carboxilic acid
XWGRWPGPPIPP

SEQ ID NO: 562 - protein, 12 aa, Bothrops jararaca
  mat_peptide (1): Xaa is a pyrrolidone carboxilic acid
XGWRWPGPPIPP

SEQ ID NO: 563 - protein, 12 aa, Bothrops jararaca
  mat_peptide (1): Xaa is a pyrrolidone carboxilic acid
XWGAWPGPPIPP

SEQ ID NO: 564 - protein, 12 aa, Bothrops jararaca
  mat_peptide (1): Xaa is a pyrrolidone carboxilic acid
XGWAWPGPPIPP

SEQ ID NO: 565 - protein, 12 aa, Bothrops jararaca
  mat_peptide (1): Xaa is a pyrrolidone carboxilic acid
XWGRPPRPPIPP

SEQ ID NO: 566 - protein, 12 aa, Bothrops jararaca
  mat_peptide (1): Xaa is a pyrrolidone carboxilic acid
XGWRPPRPPIPP

SEQ ID NO: 567 - protein, 12 aa, Bothrops jararaca
  mat_peptide (1): Xaa is a pyrrolidone carboxilic acid
XWGAPPRPPIPP

SEQ ID NO: 568 - protein, 12 aa, Bothrops jararaca
  mat_peptide (1): Xaa is a pyrrolidone carboxilic acid
XGWAPPRPPIPP

SEQ ID NO: 569 - protein, 12 aa, Bothrops jararaca
  mat_peptide (1): Xaa is a pyrrolidone carboxilic acid
XWGRWPRPPIPP

SEQ ID NO: 570 - protein, 12 aa, Bothrops jararaca
  mat_peptide (1): Xaa is a pyrrolidone carboxilic acid
XGWRWPRPPIPP

SEQ ID NO: 571 - protein, 12 aa, Bothrops jararaca
  mat_peptide (1): Xaa is a pyrrolidone carboxilic acid
XWGAWPRPPIPP

SEQ ID NO: 572 - protein, 12 aa, Bothrops jararaca
  mat_peptide (1): Xaa is a pyrrolidone carboxilic acid
XGWAWPRPPIPP

SEQ ID NO: 573 - protein, 12 aa, Bothrops jararaca
  mat_peptide (1): Xaa is a pyrrolidone carboxilic acid
XWGRPPGPZIPP

SEQ ID NO: 574 - protein, 12 aa, Bothrops jararaca
  mat_peptide (1): Xaa is a pyrrolidone carboxilic acid
XGWRPPGPZIPP

SEQ ID NO: 575 - protein, 12 aa, Bothrops jararaca
  mat_peptide (1): Xaa is a pyrrolidone carboxilic acid
XWGAPPGPZIPP

SEQ ID NO: 576 - protein, 12 aa, Bothrops jararaca
  mat_peptide (1): Xaa is a pyrrolidone carboxilic acid
XGWAPPGPZIPP

SEQ ID NO: 577 - protein, 12 aa, Bothrops jararaca
  mat_peptide (1): Xaa is a pyrrolidone carboxilic acid
XWGRWPGPZIPP

SEQ ID NO: 578 - protein, 12 aa, Bothrops jararaca
  mat_peptide (1): Xaa is a pyrrolidone carboxilic acid
XGWRWPGPZIPP

SEQ ID NO: 579 - protein, 12 aa, Bothrops jararaca
  mat_peptide (1): Xaa is a pyrrolidone carboxilic acid
XWGAWPGPZIPP

SEQ ID NO: 580 - protein, 12 aa, Bothrops jararaca
  mat_peptide (1): Xaa is a pyrrolidone carboxilic acid
XGWAWPGPZIPP

SEQ ID NO: 581 - protein, 12 aa, Bothrops jararaca
  mat_peptide (1): Xaa is a pyrrolidone carboxilic acid
XWGRPPRPZIPP

SEQ ID NO: 582 - protein, 12 aa, Bothrops jararaca
  mat_peptide (1): Xaa is a pyrrolidone carboxilic acid
XGWRPPRPZIPP

SEQ ID NO: 583 - protein, 12 aa, Bothrops jararaca
  mat_peptide (1): Xaa is a pyrrolidone carboxilic acid
XWGAPPRPZIPP

SEQ ID NO: 584 - protein, 12 aa, Bothrops jararaca
  mat_peptide (1): Xaa is a pyrrolidone carboxilic acid
XGWAPPRPZIPP

SEQ ID NO: 585 - protein, 12 aa, Bothrops jararaca
  mat_peptide (1): Xaa is a pyrrolidone carboxilic acid
XWGRWPRPZIPP

SEQ ID NO: 586 - protein, 12 aa, Bothrops jararaca
  mat_peptide (1): Xaa is a pyrrolidone carboxilic acid
XGWRWPRPZIPP

SEQ ID NO: 587 - protein, 12 aa, Bothrops jararaca
  mat_peptide (1): Xaa is a pyrrolidone carboxilic acid
XWGAWPRPZIPP

SEQ ID NO: 588 - protein, 12 aa, Bothrops jararaca
  mat_peptide (1): Xaa is a pyrrolidone carboxilic acid
XGWAWPRPZIPP

SEQ ID NO: 589 - protein, 12 aa, Bothrops jararaca
  mat_peptide (1): Xaa is a pyrrolidone carboxilic acid
XWWAWPRPZIPP

SEQ ID NO: 590 - protein, 13 aa, Bothrops jararaca
  mat_peptide (1): Xaa is a pyrrolidone carboxilic acid
XGGWPRPGPZIPP

SEQ ID NO: 591 - protein, 13 aa, Bothrops jararaca
  mat_peptide (1): Xaa is a pyrrolidone carboxilic acid
XGPWPRPGPZIPP

SEQ ID NO: 592 - protein, 13 aa, Bothrops jararaca
  mat_peptide (1): Xaa is a pyrrolidone carboxilic acid
XGGLPRPGPZIPP

SEQ ID NO: 593 - protein, 13 aa, Bothrops jararaca
  mat_peptide (1): Xaa is a pyrrolidone carboxilic acid
XGGWPHPGPZIPP

SEQ ID NO: 594 - protein, 13 aa, Bothrops jararaca
  mat_peptide (1): Xaa is a pyrrolidone carboxilic acid
XGGLPHPGPZIPP

(19) United States
(12) Patent Application Publication
(10) Pub. No.: US 2008/0199503 A1
(43) Pub. Date: Aug. 21, 2008

(54) Pharmaceutical Compositions Preparation of Peptides, Secreted by the Snake Venom Glands, Particularly of Bothrops Jararaca, Vasopeptidases Inhibitors, Evasins, Their Analogues, Derivatives and Products Associated, Thereof, for Development of Applications and Use in Chronic-Degenerative Diseases

(75) Inventors: Antonio Camargo, Sao Paulo (BR); Robson Santos, Belo Horizonte (BR); Ruben Millan, Belo Horizonte (BR); Danielle Ianzer, San Paulo (BR); Mirian Hayashi, Sao Paulo (BR)

Correspondence Address: BIRCH STEWART KOLASCH & BIRCH, PO BOX 747, FALLS CHURCH, VA, 22040-0747, US

(73) Assignee: Biolab Sanus Farmaceutica Ltd., San Paulo (BR)

(21) Appl. No.: 10/537,264

(22) PCT Filed: Dec. 9, 2003
(86) PCT No.: PCT/BR03/00192

§ 371 Date: Feb. 28, 2008

(30) Foreign Application Priority Data
Dec. 9, 2002 (BR) PI0205449-3

(51) Int. Cl. A61K 38/08; A61K 38/10; A61K 9/50; A61K 9/127; A61F 13/00; A61P 15/16; A61P 9/00

(52) U.S. Cl. 424/422; 514/17; 514/16; 514/15; 514/14; 424/493; 424/450

(57) ABSTRACT

The present invention is characterized by the process of preparation of pharmaceutical compositions for the development of applications of the Evasins and their structural and/or conformational analogues in chronic-degenerative diseases. It is further characterized by the process of preparation of pharmaceutical compositions and related products of the Evasins peptides and their structural and/or conformational analogues in using the cyclodextrins, its derivatives, liposomes and biodegradable polymers and/or mixture of these systems. The invention is further characterized by the increased efficacy of these peptides and their analogues included in cyclodextrins, when administered to rats. This characterizes an increased biodisponibility of these peptides and their analogues using the compositions of the present invention.

The present invention is characterized by the preparation process of pharmaceutical compositions of peptides secreted by the snake venom gland, particularly of the Bothrops Jararaca, vasopeptidase inhibitors peptides, Evasins, their analogues and derivatives and products associated for development of application and/or associated products for chronic-degenerative diseases.

It is further characterized by pharmaceutical compositions and/or related products of vasopeptidase inhibitors peptides, Evasins peptides and their structural and/or conformational analogues and derivatives included in cyclodextrins or their derivatives, or associated or included in carriers and/or excipientes pharmacologically acceptable. Another characteristic of this invention is the microencapsulation of Evasins their analogues and derivatives included or not in cyclodextrins, in controlled—release systems such as liposomes and biodegradable polymers and mixtures thereof.

The pharmaceutical compositions claimed in this patent comprises the Evasins included in cyclodextrins or their derivatives; or Evasins associated or included in carriers and/or pharmaceutically acceptable excipients, alone or mixed or associated at least with one pharmacologically active agent; or Evasins included or not in cyclodextrins, microencapsulated or not in controlled-release systems such us the liposomes and biodegradable polymers and/or mixtures thereof.

The present invention also comprises the identification of other biochemical mechanisms of action of Evasins having an application in the study and treatment of chronic degenerative diseases.

The pharmaceutical compositions of the Evasins, and their analogues and derivatives, except the Evasin 7a, are characterized by the differential inhibitory activity for the neutral endopeptidase (Ki in the micro-molar range) and the Angiotensin I converting enzyme (Ki—in the nano-molar range). Another characteristic of these pharmaceutical compositions there in is the increase of the biodisponibility, duration and/or efficacy of the Evasins effect when included in cyclodextrins, or their derivatives.

The pharmaceutical compositions there in present an increase of the biodisponibility, duration and/or efficacy of the Evasins included in cyclodextrins, or their derivatives, when given by oral or venous route, as no limitante example. In most of the countries worldwide, 15% to 25% of the adult population experience hypertension (NacMahon, S. et. Al. Blood pressure, stroke, and coronary heart disease, Lancet 335:765-774, 1990). The cardiovascular risk increases with the blood pressure level. The higher the blood pressure is, the higher is the risk of stroke and coronary events. Regarded as the main risk factor of coronary, cerebral and renal vascular diseases, hypertension is the main cause of death and disability among adults.

Worldwide, heart failure is the main cause of hospitalization of patients 60 to 80 years old. The population ageing itself represents a factor of increased incidence, while 1% of the individuals experience heart failure from 25 to 54 years old; among elder individuals. this incidence is much higher, reaching about 10% of those over 75 years (Kannel, W. B. et. al. Changing epidemiological features of cardiac failure, Br. Hear J 1994; 72 (suppl): S3-S9).

As per its clinical profile, heart failure is a limiting disease; when aggravated, the patients' quality of life decreases and, in more advanced cases, it has the features of a malignant disease having a mortality level above 60% in the first year, even nowadays (Oliveira, M. T. Caracteristicas clinicas e prognóstico de pacientes corn insuficência cardiaca congestiva avancada, Faculdade de Medicina, USP 1999). It is currently estimated that more than 15 million people in the industrialized world are affected; only in the USA, for example, it is estimated that the number of cases has increased 450% from 1973 to 1990 (Kannel, W. B. et. El. Changing epidemiological features of cardiac failure, Br. Hear J 1994; 72 (suppl 3): S3-S9).

Hypertension is a complex, multifactoral and highly prevalent disease, being responsible for several deleterious adverse effects and high morbidity/mortality (Kaplan, N. M. Blood pressure as a cardiovascular risk factor: prevention and treatment. JAMA. 275:1571-1576, 1996). A number of studies for evaluating the efficacy of its control in the general population and special groups have been conducted, aiming a better understanding. The pressure control without a wide non-drug and/or pharmaceutical intervention with the associated risk factors (diabetes, obesity, smoking) may reduce or even eliminate the blood hypertension long-term treatment benefits concerning mortality reduction, generally caused by coronary disease (Wilson, P. W. et. al. Hypertension, the risk factors and the risk of cardiovascular disease. Raven Press. 94-114).

Hypertension is the main contribution factor to cardiovascular arteriosclerosis (The fifth Report of the Joint National Committee on detection, evaluation, and treatment of High Blood Pressure. National Institute of Health (VJNC). Arch. Intern. Med. 153:154-181, 1994). As per the statistics, one of four Americans is or will be a hypertensive patient; 4.78 million are estimated to experience heart failure. Four hundred thousand new cases are diagnosed every year, leading to 800 thousand hospitalizations; US$ 17.8 billion dollars are spent with treatment.

In Brazil, data from SUS [Unified Health System] show that heart failure was the main cause of hospitalizations among the cardiac diseases in 1997; the government spent R$ 150 millions in treatment, this amount being equivalent to 4.6% of the health expenses (Filho, Albanesi F. Insuficiência cardiaca no Brasil. Arq. Bras. Cardiol, 71:561-562, 1998).

The renin-angiotensin system (RAS) is responsible for regulating the blood pressure, cardiovascular homeostasis and hydroelectrolytic balance, both under physiological and pathological conditions (Santos, R. A. S.; Campagnole-Santos, M. J.; Andrade, S. P. Angiotensin-(1-7): an update. Regulatory Peptides, 91:45-62, 2000). Angiotensin II (Ang II) is the main effector peptide of RAS, having vasopressor, adrenal steroids synthesis stimulating, proliferating (fibroblasts, vascular smooth muscle) and hypertrophic (cardiac myocites) actions. Its formation pathway involves the production of angiotensinogen by the liver and the production of renin in the justaglomerular system. These substances are released in the blood stream where the angiotensinogen is hydrolyzed by renin, thus forming Ang L that, when in the lungs, will undergo the action of the angiotensin converting enzyme (ACE) and generate Ang II. Ang II, in turn, will act on target organs remote from its production site (Santos, R. A. S.; Campagnole-Santos, M. J.; Andrade, S. P. Angiotensin-(1-7): an update. Regulatory Peptides, 91:45-62, 2000).

It was recently discovered that, in addition to the system that generates the circulating Ang II, different tissues contain independent Ang II generating RAS, apparently by local action. The tissue RAS components are found on the walls of blood vessels, uterus, exocrine portion of pancreas, eyes, heart, adrenal cortex, testis, ovaries, pituitary gland anterior and intermediate lobes, pineal and brain. The functions of these tissue SRAs are not very clear yet. (Ardaillou, R.; Michel, J. B. The relative roles of circulating and tissue renin-angiotensin systems. Nephrol. Dial. Transplant., 14:283-286, 1999). The local actions of RAS may occur at the cell producing peptides (intracrine and autocrine functions), on adjacent cells (paracrine function) or in a location away from the production site (endocrine function).

Recent remarks indicate that important peripheral and central actions of RAS can be mediated by smaller angiotensinergic peptides sequences, including Ang III [Ang-(2-8)], Ang IV [Ang-(3-8)] and Ang-(1-7). We can consider that both Ang I [Ang-(1-10)] and Ang II [Ang-(1-8)] can undergo a biotransformation process, thus giving raise to a “family” of biologically active angiotensin peptides. (Santos, R. A. S.; Campagnole-Santos, M. J.; Andrade, S. P. Angiotensin-(1-7): an update. Regulatory Peptides, 91:45-62, 2000).

Ang-(1-7), together with Ang II, are the main RAS effectors. Two important characteristics segregate Ang-(1-7) from Ang II: the first has highly specific biologic actions and its formation pathway is independent from ACE (Santos, R. A. S.; Campagnole-Santos, M. J.; Andrade, S. P. Angiotensin-(1-7): an update. Regulatory Peptides, 91:45-62, 2000). Evasins would favor the formation of Ang-(1-7) by increasing Ang I concentration and reduce its metabolism by inhibiting ACE.

The primary purpose of the treatment of hypertension is not only to reduce the expenses, but also to prevent injuries in target organs by changing the quality of life and the use of drugs when required (The Fifth Report of The Joint National Committee on detection, evaluation, and treatment of High Blood Pressure. National Institute of Health (VJNC). Arch. Intern. Med. 153:154-181, 1994).

The drug treatment is indicated in cases of non-response to lifestyle changes after a term from three to six months and in case of injuries in target organs (left ventricular hypertrophy, myocardial ischemia, stroke or hypertensive retinopathy). All patients showing a systolic blood pressure above 160 mmHg or diastolic blood pressure above 100 mmHg should be subject to pharmacological treatment, regardless of other factors whether present or not (Report the Canadian Hypertension Society. Consensus Conference. 3. Pharmacological treatment of essential hypertension. Xan. Med. Assoc. J. 149 (3): 575-584, 1993).

During the 70's and 80's, however, the antihypertensive drugs became an important tool in the treatment of high blood pressure (Ménard, J. Anthology of renin-angiotensin system: A one hundred reference approach to angiotensin II antagonist. J. Hypertension 11 (suppl 3): S3-S11, 1993). During the last four decades, the pharmacological research produced new classes of drugs to treat hypertension: diuretics during the 60's, beta-blockers in the 70's, calcium channel blockers, antagonists of angiotensin II receptors and angiotensin converting enzyme (ACE) inhibitors.

Diuretics can be divided into three categories: thiazides loop and potassium sparing. Thiazides and the like include Chlorothiazide and Hydrochlorothiazide that reduce the blood pressure in about 10 to 15% within the first days of treatment, this reduction being related to a deceased secondary extracellular volume and increased diuresis and natriuresis. Then, after six months, plasma volume and cardiac output return to normal values and the decreased blood pressure is related to a decreased peripheral vascular resistance (Frolich, E. Current approaches in the treatment of hypertension, 405-469). They are often used as a monotherapy, showing improved responses in black patients and, at low doses, in elder patients. The following side effects are seen: increased peripheral resistance to insulin, increased triglycerides, increased LDL, hypocalcemia, hyperuricemia Furosemide, Bumetamide and Trianterene are among the loop diuretics showing higher potency than the thiazide diuretics. They primarily act on medullar and cortical portions of the Henle's loop. They show the same side effects of thiazide diuretics. The potassium sparing are drugs having a weak diuretic action, being rarely used alone. Among them, Amiloride, Trianterene and Spirolactone can be cited.

Beta-blockers, including Atenolol and Naolol, are classified as beta-1 and beta-2. The mechanism of antihypertensive action is not completely clear yet; however, it is basically supported by evidences that beta-blockers inhibit the presynaptic beta receptors, thus preventing the release of noradrenalin. The side effects include: change in the response to insulin, hypoglucemic coma extension, increased triglycerides and increased creatinine by reducing the renal flow.

The calcium channel blockers have been used for at least 25 years (Frolich, E. D. Current Approaches in the Treatment of Hypertension, 405-469, 1994). They can be divided into two large groups according to their pharmacological actions: those having increased action on the stimulus conduction, such as Verapamil and Diltiazem and those having a predominant vasodilator action, such as the dihydropiridine derivatives (Nifedipine and others) (Frolich, E. D., Hypertension. Adult Clinical Cardiology Self Assessment Program (ACCSAP), 6: 3-19, 1995). Side effects include edema of lower limbs and tachycardia.

The converting enzyme inhibitors primarily act by inhibiting the conversion of angiotensin I to angiotensin II. Thus, essentially vasoconstricting actions of angiotensin II are minimized. Preliminary studies show that teprotide (Evasin-9a), the first clinically used inhibitor has an antihypertensive activity when administered intravenously, however it is inactive when given orally, thus limiting its use. We currently know that ACE is an enzyme with multiple actions, that is, it acts on several substrates. In addition to act as a dipeptidase on angiotensin I and bradykinin, it is also able to disrupt peptide chains of the natriuretic peptide, indicating that the enzyme can act on several tissues. ACE has also an important role in the circulating and tissue Ang-(1-7) inactivation. The concentration of this circulating peptide is similar to the concentration of Ang II and is increased after the ACE inhibition. This increase can be a result of the increased precursor (Ang I) and decreased ACE-degradation (Santos, R. A. S.; Campagnole-Santos, M. J.; Andrade, S. P. Angiotensin-(1-7): an update. Regulatory Peptides, 91:45-62, 2000).

The following patents were found in the state of the art that refer to the omapatrilat activity: US2002013307-A1, Kothari and Desai; US2002004500-A1 (WO200174348-A2, AU200187289-A), Bristol-Myers Squibb Co (BRIM) and Reeves et al.; U.S. Pat. No. 6,166,227-A (WO200003981-A2, AU9948528-A), Bristol-Myers Squibb Co(BRIM) and Godfrey et al. However, this vasopeptidase inhibitor compound and their analogs show relevant side effects (angioedema and cough) as a result of the high inhibitory activity on the neutral endopeptidase (NEP).

The ACEI are excellent when administered as a monotherapy, since the ACE inhibitors lead to a relatively fast reduction of the blood pressure in 60 to 70% of the hypertensive patients (Ganong, W. Neuropeptides in cardiovascular control. J. Hypertens 2 (suppl 3): 15-22, 1984). They are generally well-tolerated, however their use may cause side effects and adverse reactions, some of them relatively severe and including angioneurotic edema, rashes and dry cough (8 to 10%), blood discrasias and sexual impotence.

The Angiotensin I converting enzyme inhibitors or the vasopeptidase inhibitors have been used or proposed to preventing or treating many diseases including tumors, acute myocardial infarction, stroke, left ventricular hypertrophy, diabetes angiopathy, peripheral ischemia, angina and progression of the heart failure after myocardial infarction and atherosclerose, mellitus diabetes and angiogenesis. (Yasumaru M, Tsuji S, Tsujii M, Irie T, Komori M, Kimura A, Nishida T, Kakiuchi Y, Kawai N, Murata H, Horimoto M, Sasaki Y, Hayashi N, Kawano S, Hori Inhibition of angiotensin II activity enhanced the antitumor effect of cyclooxygenase-2 inhibitors via insulin-like growth factor I receptor pathway; Cancer Res. 2003 Oct. 15; 63(20):6726-34; Kinuya S, Yokoyama K, Kawashima A, Hiramatsu T, Konishi S, Shuke N, Watanabe N, Takayama T, Michigishi T, Tonami N. Pharmacologic intervention with angiotensin II and kininase inhibitor enhanced efficacy of radioimmunotherapy in human colon cancer xenografts. J Nucl Med. 2000 July; 41(7): 1244-9; Volpert O V, Ward W F, Lingen M W, Chesler L, Solt D B, Johnson M D, Molteni A, Polyerini P J, Bouck N P. Captopril inhibits angiogenesis and slows the growth of experimental tumors in rats. J Clin Invest. 1996 Aug. 1; 98(3):671-9; Kowalski J, Herman Z S. Captopril augments antitumor activity of cyclophosphamide in mice. Pol J. Pharmacol. 1996 May-June; 48(3):281-5; Kowalski J, Belowski D, Madej A, Herman Z S. Effects of thiorphan, bestatin and captopril on the Lewis lung carcinoma metastases in mice. Pol J. Pharmacol. 1995 September-October; 47(5):423-7; Kowalski J, Belowski D, Wielgus J, Gabryel B, Klin M, Herman Z S. Effect of captopril and thiorphan on the proliferation of human neoplastic cell lines and their influence on cytostatic activity of interferon alpha or cytotoxic activity of doxorubicin. Arch Immunol Ther Exp (Warsz). 1995; 43(1):47-50. However, it was not found in the state of the art any patent that claim the pharmaceutical composition of the Evasins included in cyclodextrins or not, formulated with carriers or excipients pharmacologically acceptable with application in the study and treatment of degenerative chronic diseases.

The first attempts to develop Ang II antagonists are dated from the beginning of the 70's and were focused on the development of Ang II-analog peptides. The first, saralasin, 1-sarcosine, 8-isoleucine angiotensin II and then others. However, they were not clinically accepted, since they showed partial agonist activity. In 1982, the first two AT₁ receptors and non-peptide antagonists were developed (S-8307 and S-8308) and, although having a highly specific and no agonist activity, showed a weak binding to Ang II receptors. After several changes in the molecular structure of these two parent compounds aiming to improve strength, keep selectivity and reach pharmacokinetic properties, a new, potent and high-specificity oral product was developed, that is, Losartan. Since then, several other non-peptide antagonists were developed, such as, Candesartan, Irbesartan, Valsartan, Teimisartan, Eprosartan, Tasosartan and Zolasartan. The pharmaceutical compositions and formulations of the present invention characterized by the use of a mixture of the pharmaceutically combined and acceptable excipients Evasins and analogs. Formulations can be prepared with an excipient or mixtures thereof. Examples of excipients include water, saline, phosphate-buffered solutions, Ringer's solution, dextrose solution, Hank's solution, biocompatible saline solutions whether containing polyethylene glycol or not. Other useful formulations include viscosity-increasing agents, such as sodium carboxymethylcellulose, sorbitol or dextran. The excipients can also contain lower amounts of additives, such as substances that increase the isotonicity and chemical stability or buffers. Examples of buffers include phosphate buffer, bicarbonate buffer and Tris buffer; examples of preservatives include thimerosal, meta- or ortho-cresol, formalin and benzyl alcohol. Standard formulations can be liquid or solid. Thus, in a non-liquid formulation, the excipient may include dextrose, human serum albumin, preservatives, etc, to which water or sterile saline can be added before the administration.

The present invention is further characterized by the preparation of controlled-release systems containing Evasins their analogs and derivatives. The satisfactory controlled-release systems include, but are not limited to cyclodextrins, biocompatible polymers, biodegradable polymers, other polymeric matrixes, capsules, microcapsules, microparticles, bolus preparations, osmotic pumps, diffusion devices, liposomes, lipospheres and transdermal administration systems. Other controlled-release compositions of the present invention include liquids that, after being administered to an animal, form a solid or gel in situ.

U.S. Pat. No. 4,598,070 (CA1215359, DK356684, EP135044, ES8506757, GR82322, JP60025967). Mashiro, Kawahara et. al. (1986) developed an invention referring to inclusion compounds between Tripudine (an antihypertensive) and cyclodextrins α-cyclodextrin and β-cyclodextrin). Tripamide is slightly soluble in water and, thus, the use of cyclodextrins allowed more soluble compounds to be obtained. This invention also employed some characterization techniques, such as: differential scanning calorimetry (DSC) and X-rays diffraction.

U.S. Pat. No. 4,666,705. De Crosta, Mark. T. et. al. (1987) reported an invention based on the controlled release of hypertension drugs. Captopril, an ACE inhibitor (non-peptide, active site-directed ACE inhibitor) was used, since this inhibitor shows fast absorption and a half-life of 2 hours. So as to retard the release, this invention was based on tablets containing captopril together with the polymer or copolymer. The polymer used was polyvinyl pyrrolidone (PVP) and dry granulation was the technique. The result obtained was an increased indwelling time of the drug in the body (4 to 16 hours).

U.S. Pat. No. 5,519,012. Fercej-Temeljoov, Darja et. al. (1996) developed an invention based on a new inclusion compound of a antihypertensive, that is, 1,4-dihydropyridine, with methyl-(β-cyclodextrin and other derivatives, such as (hydroxylated β-cyclodextrin).

U.S. Pat. No. 5,728,402. Chen, Chih-Ming et. al. (1998) disclosed an invention based on the controlled release of drugs by means of a pharmaceutical composition containing an inner phase comprising captopril and hydrogel, and another outer insoluble phase in the stomach. This invention intended to increase the drug absorption time that, when administered alone, is 1 hour.

U.S. Pat. No. 5,834,432 (AU5990796, CA2221730, EP0828505, WO09639164, JP115073625). Rodgers, Katlen Elizabeth et. al. (1998) used AT2 receptors agonists to accelerate wound healing.

U.S. Pat. No. 6,087,386 (WO9749392A1). Chen, Tzyy-Show R et. al. (2000) disclosed invention consisting in a pharmaceutical composition containing Enalapril (ACE inhibitor) and Losartan (AII antagonist), comprising a layer of Losartan Potassium and another layer of Enalapril Maleate. This invention intended to improve the pharmacological action, reduce side effects and increase the absorption time.

U.S. Pat. No. 6,178,349. Kieval, Roberts S. et. al. (2001) developed a device based on the release of drugs by means of neural stimulation for the treatment of cardiovascular diseases. This device comprises an electrode connected to the nerve, an implantable pulse generator and a reservoir containing the drug to be administered. During use, the electrode and the drug release stimulates the nerve that, in turn, affects the control on the cardiovascular system.

A drug can be chemically modified so as to release its properties, such as biodistribution, pharmacokinetics and solubility. Several methods have been used to increase the drugs' solubility and stability, including the use of organic solvents, emulsions, liposomes, pH adjustment, chemical changes and drugs complexation with an appropriate encapsulating agent, such as cyclodextrins, liposomes and microencapsulation in biodegradable polymers.

Cyclodextrins were first isolated in 1891 by Vilers, as the starch degradation products by the action of amylase of Bacillus macerans. In 1904, Schardinger characterized them as cyclic oligosaccharides. In 1938, Frudenberg et al reported that cyclodextrins comprise glucose units joined by (α1→4) binding. In 1948, Freudenberg and colleagues observed that cyclodextrins are able to form inclusion compounds or complexes and, latter, such as French et al, prepared synthesis processes of pure cyclodextrins. From 1954, Cramer et al conducted a systematic study on the formation of cyclodextrins complexes with other compounds. From 1955 to 1960, the first studies on the formation of cyclodextrins inclusion complexes with drugs were conducted. These studies are intensively continued in Japan, Hungary, France, Italy and other countries.

Cyclodextrins are obtained by the enzymatic degradation of starch. The methods comprise the following phases: enzyme production and purification, enzymatic transformation of starch and recovery and separation of cyclodextrins. The enzyme involved is a cyclodextrin-glycosyltransferase (CGT). This enzyme is obtained from several microorganisms, but mainly Bacillus macerans, B. megatherium, B. stereothermophilus and Klebsiella pneumoniae. (Korollkovas, A. Inclusäo molecular e cyclodextrinas: propriedades e aplicaçöes terapêuticas. ENLACE Farmalab, 2/91, Ano 5, Vol. II, p. 6-15).

Cyclodextrins are cyclic oligosaccharides that include six, seven or eight glucopiranose units. As a result of steric interactions, the cyclodextrins, CDs, form a cyclic structure in the shape of a truncated cone having an internal apolar cavity. It includes chemically stable compounds that can be region-selectively modified. The cyclodextrins (hosts) form complexes with several hydrophobic molecules (guest matter), completely or partially including them in the cavity. The CDs have been used for the solubilization and encapsulation of drugs, perfumes and aromatic agents as described by Szejtli, J., Chemical Reviews, (1998), 98, 1743-1753. Szejtli, J., J. Mater. Chem., (1997), 7, 575-587. According to detailed toxicity, mutagenicity, teratogenicity and carcinogenicity studies on cyclodextrins, described in [Rajewski, R. A., Stella, V., J. Pharmaceutical Sciences, (1996), 85, 1142-1169], they have a low toxicity, especially the hydroxypropyl-(-cyclodextrins, as reported in Szejtli J. Cyclodextrins: Properties and applications. Drug Investig., 2 (suppl. 4): 11-21, 1990. Except for high concentrations of some derivatives that damage erythrocytes, these products generally impose no health risks. The use of cyclodextrins as additives in foodstuff has been authorized in countries such as Japan and Hungary and for more specific applications in France and Denmark. All these characteristics mean an increasing motivation for the discovery of new applications.

In addition to cyclodextrins, biodegradable polymers are also used, which decrease the absorption speed of the drugs in the body by means of the controlled-release devices. In these systems, the drugs are incorporated in a polymeric matrix based in the encapsulation of the drugs in microspheres or nanospheres that release the drug inside the body in small and controllable daily doses for days, months or even years.

Several polymers have already been tested in controlled-release systems. Several of them as a function of their physical properties, such as: poly(urethanes) due to its elasticity, poly(siloxanes) or silicone for being a good isolating agent, poly(methyl-methacrylate) as a result of its physical strength, poly(vinyl alcohol) as a result of its hydrophobicity and resistance, poly(ethylene) by virtue of its hardness and impermeability (Gilding, D. K. Biodegradable polymers. Biocompat. Clin. Implat. Mater. 2:209-232, 1981).

Nevertheless, for use in humans, the material should be chemically inert and free of impurities. Some of the materials used on release systems include: poly(2-hydroxy-ethylmethacrylate), polyacrylamide, lactic acid-based (PLA) and glycolic acid-based (PGA) polymers and the corresponding copolymers, (PLGA) and the poly (anhydrides), such as the sebasic acid-based (PSA) polymers and copolymers with hydrophobic polymers.

The prior art relates several patents for the preparation of liposomes [U.S. Pat. No. 4,552,803, Lenlc; U.S. Pat. No. 4,310,506, Baldeschwieler; U.S. Pat. No. 4,235,871, Papahadjopoulos; U.S. Pat. No. 4,224,179, Schneider; U.S. Pat. No. 4,078,052, Papahadjopoulos; U.S. Pat. No. 4,394,372, Alfaiate; U.S. Pat. No. 4,308,166, Marchetti; U.S. Pat. No. 4,485,054, Mezei; and U.S. Pat. No. 4,508,703, Redziniak; Woodle and Papahadjopoulos, Methods Enzymol. 171:193-215 (1989)].

Unilamellares liposomes have a single membrane that contains an aqueous volume [Huang, Biochemistry 8:334-352 (1969)], while the multilamellar liposomes have several concentrical membranes [Bangham et Col., J. Mol. Biol. 13:238-252 (1965)]. Liposomes-based carriers were proposed for a variety of pharmacologically active substances, including antibiotics, hormones and antitumor agents [Medical applications of liposomes (D. D. Lasic, D. Papahadjopoulos Ed.), Elsevier Science B.V., Holland, 1998].

Other preparation processes of liposomes have been found in the prior art, [for a review, see, for example, Cullis et al., in: Liposomes, From Biophysics to Therapeutics (M. Ostro, ed.), Marcel Dekker (New York), 1987, pp. 39-72; Woodle and Papahadjopoulos, Methods Enzymol. 171:193-215 (1989); Liposome technology (G. Gregoriadis ed.), CRC Press, Boca Raton, Fla., 1993).

The Bangham's procedure [J. Mol. Biol. 13:238-252 (1965)] produces “ordinary multilamellar liposomes” MLVs). The “ordinary” MLVs can have an unequal solute distribution among the aqueous compartments and thus show an osmotic pressure differential among the compartments. Lenk et Col. (U.S. Pat. No. 4,522,803; U.S. Pat. No. 5,030,453 and U.S. Pat. No. 5,169,637), Fountain et al. (U.S. Pat. No. 4,588,578), Cullis et al. (U.S. Pat. No. 4,975,282) and Gregoriadis et al. (Patent WO99/65465) discovered methods for the preparation of multilamellar liposomes having a substantially equal solute distribution among the compartments. An equal solute distribution among the different compartments means a greater drug encapsulation efficacy, as well as a lower osmotic pressure differential, then rendering these MLVs more stable than the ordinary MLVs.

Unilamellar liposomes can be produced by sonication of MLVs [see Paphadjopoulos et al. (1968)] or by extrusion through polycarbonate membranes [Cullis et Col. (U.S. Pat. No. 5,008,050) and Loughrey et Col. (U.S. Pat. No. 5,059,421)]. Satisfactory lipids include, for example, phosphatidylcholine, phosphatidylserine, phosphatidylglycerol, cardiolipin, cholesterol, phosphatidic acid, sphingolipides, glycolipids, fatty acids, sterols, phosphatidylethanolamine, polymerizable phospholipids in their declared polymerized or non-polymerized form, and mixtures thereof.

The liposomes compositions of the present invention are characterized by a modification of vesicles, thus providing specificity to organs or cells. Liposomes direction was classified based on anatomical factors and mechanisms involved. The anatomical classification is based on the selectivity level, for example, organ-specific, cell-specific or organelle-specific. From the mechanisms point of view, directing can be ranked as passive or active.

Passive directing uses the natural trend of conventional liposomes to be captured by cells of the reticuloendothelial system in organs containing sinusoidal capillaries. The liposomes of the present invention are sterically established by the (LEE) method (also known as “PEG-liposomes”) as an improved drug carrier, due to its reduced elimination speed into blood stream [Lasic and Martin, Stealth Liposomes, CRC Press, Inc., Boca Raton, Fla. (1995)]. LEE are liposomes, the surface of which is covered by a polymer, this polymer being preferably polyethylene glycol (PEG), covalently conjugated to one of the phospholipids and generates a hydrophilous cloud out of the vesicle double layer. This steric barrier retards the liposomes recognition by opsonins and allows LEE to remain in the blood stream for a longer time than the conventional liposomes [Lasic and Martin, Stealth Liposomes, CRC Press, Inc., Boca Raton, Fla. (1995); Woodle et Col., Biochim. Biophys. Acta 1105:193-200 (1992); Litzinger et Col., Biochim. Biophys. Acta 1190:99-107 (1994); Bedu Addo, et Col., Pharm. Res. 13:718-724 (1996)] and increase the pharmacological efficacy of encapsulated agents, as showed for some chemotherapeutic drugs (Lasic and Martin, Cautela Liposomes, CRC Press, Inc., Boca Raton, Fla. (1995)] and bioactive peptides [Allen T. M. In: Liposomes, New Systems, New Trends in their Applications (F. Puisieux, P. Couvreur, J. Delattre, J. -P. Devissaguet Ed.), Editions de la Santé, Franca, 1995, pp. 125].

Studies in this field demonstrated that different factors affect the circulation half-life of LEE and, ideally, the vesicles diameter should be lower than 200 nm, the PEG having a molecular weight of about 2,000 Da at a ratio of 5% [Lasic and Martin, Cautela Liposomes, CRC Press, Inc., Boca Raton, Fla. (1995); Woodle et Col., Biochim. Biophys. Acta 1105:193-200 (1992); Litzinger et Col., Biochim. Biophys. Acta 1190:99-107 (1994); Bedu Addo et Col., Pharm. Res. 13:718-724 (1996)].

Active directing involves the change of liposomes by means of its association with a binder, such as a monoclonal antibody, sugar, glycolipid, protein, polymer or by changing the composition or size of liposomes to direct them towards organs and cells different from sites where the conventional liposomes accumulate. See, for example, Remington's Pharmaceutical Sciences, Gannaro, A. R., ed., Mack Publishing, 18th edition, pp. 1691-1693.

An example found in the state of the liposomes application technical to prolong the effect of the peptide is the preparation of the liposomes containing Ang-(1-7) (LAng) unilaterally microinjected (in the rostro-ventrolateral medulla (RVL. The blood pressure was measured by telemetry for 10 seconds, every 10 minutes, starting 4 days before and finishing 12 days later in undisturbed rats with free movement. The LAng microinjection produced a significant pressor effect during the morning period and maintained for 5 days. The highest MBP was measured on day 3 (114±4 mmHg) that was significantly different from that measured on day 0 (100±3 mmHg). As expected, the Lvaz did not change significantly in MBP (94±5 mmHg on day 3 vs 90±5 mmHg on day 0). Additionally, morning MBP was significantly higher in the Lang group than in the Lvaz group on days 1, 2 and 3. Contrary to the morning MBP, the night MBP was not significantly affected by the LAng microinjection. Previous studies (Fontes M A, Pinge M C, Naves V, Campagnole-Santos M J, Lopes O U, Khosla M C, Santos RAS Cardiovascular effects produced by microinjection of angiotensins and angiotensin antagonists into the ventrolateral medulla of freely moving rats. Brain Res. 1997 Mar. 7; 750(1-2):305-10) established that the free Ang-(1-7) microinjection (not encapsulated) on BRVL at a similar dose (25 to 50 ng), leads to an increase of 15 mmHg for approximately 10 min. The short length of this effect was due to the peptide high metabolism in vivo [Silva-Barcellos et Col., Hypertension, 38(6): 1266-71 (2001).

The present invention is characterized by the use of at least twenty-one bradykinin potentiating peptides found in the venom and tissues of Bothrops jararaca (generically called BPPs or bradykinin potentiating peptides) had their amino acids sequences determined by mass spectrometry or deduced in cDNA of parent compounds of these molecules expressed in non-venom gland tissues of this serpent (called Evasins or Endogenous Vasopeptidase Inhibitors).

| | |
|---|---|
| Nomenclature | Sequence |

| | | |
|---|---|---|
| ID 1 | EVASIN-5a | <EKWAP |
| ID 2 | EVASIN-5b | <EWPRP |
| ID 3 | EVASIN-5c | <EKFAP |
| ID 4 | EVASIN-6a | <ESWPGP |
| ID 5 | EVASIN-7a | <EDGPIPP |
| ID 6 | EVASIN-9a | <EWPRPQIPP |
| ID 7 | EVASIN-9b | <ESWPGNIPP |
| ID 8 | EVASIN-10a | <ESWPGPNIPP |
| ID 9 | EVASIN-10b | <ENWPRPQIPP |
| ID 10 | EVASIN-10c | <ENWPHPQIPP |
| ID 11 | EVASIN-10d | <ESWPEPNIPP |
| ID 12 | EVASIN-11a | <EWPRPTPQIPP |
| ID 13 | EVASIN-11b | <EGRAPGPPIPP |
| ID 14 | EVASIN-11c | <EGRAPHPPIPP |
| ID 15 | EVASIN-11d | <EGRPPGPPIPP |
| ID 16 | EVASIN-11e | EARPPHPPIPP |
| ID 17 | EVASIN-12a | <EGWAWPRPQIPP |
| ID 18 | EVASIN-12b | <EWGRPPGPPIPP |
| ID 19 | EVASIN-13a | <EGGWPRPGPEIPP |
| ID 20 | EVASIN-13b | <EGGLPRPGPEIPP |
| ID 21 | EVASIN-13c | <EGGWPRPGPQIPP |

Most of these peptides have the structural motif C-terminal PX¹X²PP, wherein X¹ can be any amino acid and X² is typically a residue of isoleucine (1) and the N-terminal amino acid is blocked, usually by the presence of a residue of pyroglutamic acid (<E). The corresponding synthetic peptides were tested as C and N site inhibitors of recombinant ACE and as potentiators both of the bradykinin contractile activity in guinea pig isolated ileum and the hypotensive activity of bradykinin in rats. The most selective and effective as potentiators of the bradykinin contractile action in guinea pig isolated ileum and the hypotensive action on blood pressure in rats were those having masses between 500 and 1700 Daltons, containing 5 to 13 amino acids residues. The active molecules were chemically modified, thus giving raise to other peptides having qualitatively similar characteristics.

The Evasins, oligopeptides of 5 to 13 amino acids, formulated therein, are those described below:

| | | |
|---|---|---|
| Formulas | Sequences | Nomenclature |

| | | |
|---|---|---|
| I | <E¹aa²aa³aa⁴P⁵ | Evasin-5a, b, . . . , n |
| II | <E¹aa²aa³aa⁴aa⁵P⁶ | Evasin-6a, b, . . . , n |
| III | <E¹aa²aa³aa⁴aa⁵P⁶ | Evasin-7a, b, . . . , n |
| | P⁷ | |
| IV | <E¹aa²aa³P⁴aa⁵aa⁶ | Evasin-8a, b, . . . , n |
| | P⁷P⁸ | |
| V | <E¹aa²aa³aa⁴aa⁵aa⁶ | Evasin-9a, b, . . . , n |
| | aa⁷P⁸P⁹ | |
| VI | <E¹aa²aa³aa⁴aa⁵P⁶ | Evasin-10a, b, . . . , n |
| | aa⁷aa⁸P⁹P¹⁰ | |
| VII | <E¹aa²aa³aa⁴aa⁵aa⁶ | Evasin-11a, b, . . . , n |
| | P⁷aa⁸aa⁹P¹⁰P¹¹ | |
| VIII | <E¹aa²aa³aa⁴aa⁵aa⁶ | Evasin-12a, b, . . . , n |
| | aa⁷P⁸aa⁹aa¹⁰P¹¹ | |
| | P¹² | |
| IX | <E¹aa²aa³aa⁴aa⁵aa⁶ | Evasin-13a, b, . . . , n |
| | aa⁷aa⁸P⁹aa¹⁰aa¹¹ | |
| | P¹²P¹³ | |

wherein:

P is always proline. The others could be L- or D-amino acids and derivatives that are presented with the code of three and one letters

aspartic acid (Asp, D) glutamic acid (Glu, E)
alanine (Ala, A) arginine (Arg, R)
asparagine (Asp, D) phenylalanine (Phe, F)
glycine (Gly, G) glutamine (Gln, Q)
histidine (H is, H) isoleucine (Ile, I)
leucine (Leu, L) lysine (Lys, K)
proline (Pro, P) serine (Ser, S)
tyrosine (Tyr, Y) threonine (Thr, T)
tryptophan (Trp, W) valine (Val V)
aminobutyric acid (Abu) aminoisobutyric acid (Aib)
diaminobutanoic acid (Dab) diaminopropionic acid (D)pr)
hexanoic acid (ε-Ahx) isonipecotic acid (Isn)
pyroglutamic acid Cyr, <E)
tetrahydroisoquinoline-3-carboxylic acid (Tic)
butyl-glycinecyclohexylalanine (Cha)
citrulline (Cit) statin and derivatives (Sta)
phenylglycine Phg) hydroxyproline (Hyp)
homoserine (Hse) norleucine (Nle)
norvaline (Nva) ornitin (Orn)
penicillinalanine (en) sarcosine (Sar)
iethylalanine (Thi) [SIC]

<E¹ pyroglutamic acid is the N-terminal amino acid;

aa² is an amino acid, typically W, S or K for formulas I and II, typically D for formula III and typically A, W, S, G or N for formulas IV to IX;

aa³ is typically W, P, F or G for formulas I to III and typically A, P, G, W or R for formulas IV to IX;

aa⁴ is an amino acid, typically P, A or R for formulas I to III and typically P, L, Q, A, R or W for formulas IV to IX;

aa⁵ is an amino acid, typically G, R or I for formulas II and III and typically T, P, G, H, R, W or E for formulas IV to IX;

aa is an amino acid, typically Q, N, P, T, H, R or G for formulas V, VII, VIII and IX; it is usually I, A, T or Y for formula IV;

aa⁷ is an amino acid, typically P, N, Q, G or R for formulas VI, VI and IX and usually I, A, T or Y for formula V;

aa⁸ is an amino acid, typically Q, P or G for formulas VII and IX; it is usually I, A, T or Y for formula VI;

aa⁹ is an amino acid, typically P, Q, N or G for formula VII and usually L A, T or Y for formula VII;

aa¹⁰ is an amino acid, typically Q and E for formula IX and usually I, A, T or Y for formula VIII;

aa¹¹ for formula IX is usually I, A, T or Y;

Another feature of the present invention is the possibility of a modification of all EVASINs molecules aiming to improve their pharmacokinetic and action specificity properties on different target molecules involved in cardiovascular pathologies, both as vasopeptidases inhibitors and as an action on endothelial cells and vessels smooth muscle by means of chemicals changes consisting in:
1) Localized conformational changes by substituting the L-amino acids with D-amino acids, leading to the introduction of a reverse β-“turn” structure (“hairpin”), or by introducing α-substituted amino acids, thus rendering the polypeptide chain axis intro a α-helix structure or extended conformation or β-“turn”, for example, the introduction of the α-aminoisobutyric. It is also provided a N-methyl substitution of a α-amino acid, thus restricting the action of the amide bond, eliminating the formation of a hydrogen bond, affecting the main polypeptide chain angle of torsion and allowing the formation of a cis peptide bond. Another modification is the peptide amide bond substitution with a non-amide covalent bond so as to protect this bond against the action of proteases.
2) Global Conformational Changes by cyclization, thus stabilizing secondary structures. The two amino acids chosen for the substitution with cysteine or other organic compounds containing, for example, a thiol group each with a β,β-dimethylpenicillinalanine analog, can be any of the amino acids residues of the EVASINs sequences or their analogs, being separated from each other by at least two polypeptide chain amino acids residues. The formation of a S—S bond is then favored between the two thiol residues, for example, thus forming a cyclic peptide. Cyclization can be also obtained by forming a lactam bond or peptide bond between the polypeptide free carboxylic and amino groups or any other chemical procedure that favors the peptide cyclization.
3) Changes in amino acids side chains (χ-“constraints”). The determination of the α-amino acids side chains angles of torsion may allow the topological changes that better adjust the polypeptide to its binding site, for example, the substitution of tyrosine with β-methyl-2′,6′-dimethyltyrosine (TMT) that can define a preferential conformation to the interaction site. The proposed changes were referred and exemplified in the review of Victor J. Hruby published in Nature, 1, 847-858, 2002.
The present invention is characterized by obtaining oligopeptides release systems, EVASINs, using cyclodextrins and their derivatives that reduce the degradation by the gastrointestinal tract (GIT), leading to increased bioavailability of the peptide in the biologic system particularly for orais formulations. Additionally, there are others applications forms like; intravenous, intramuscularly, topical, pulmonary inhalation, intranasal, intramounth or as a controlled liberation diapositive using biodegradable polymers as PLA and PLGA or mixture of these examples no limitants.

The present invention is further characterized by controlled-release systems of oligopeptides, Evasins, using the liposomes that increase the peptide bioavailability. Liposomes are lipid vesicles that include internal aqueous compartments in such molecules, for example, drugs can be encapsulated aiming to reach a slow drug release after the administration of the liposome to an individual.

No application using oligopeptides, Evasins, or their structural and/or conformational analogs included in cyclodextrins or their derivatives, microencapsulated into biodegradable polymers such as PLA ou PLGA ou mixed and the liposomes, was previously described. The present invention is characterized by the use of three different technologies, that is, molecular encapsulation of oligopeptides, Evasins, and their analogs into cyclodextrins and the microencapsulation into biodegradable polymers or liposomes and/or mixtures thereof allowing the increase of the biodisponibility of the Evasins in the oral compositions and formulations when compared to not formulated.

None pharmaceutical composition and/or formulation of the Evasins and their structural analogues and/or conformational characterized by the utilization of the Evasins 7a, 10c, 11e, 12b and their respectives analogues and derivatives as molecular models for development of drugs and/or pharmaceutical composition or formulations based on peptide compound and/or no-peptide was previously described.

Another feature of this invention is the use of the pharmaceutical compositions and/or formulations of the Evasins and their analogues and derivatives characterized by inclusion and/or association compounds among the Evasins and their analogues and derivatives cyclodextrins, their derivatives, microencapsulate or not in controfled-release systems such as liposomes and the biodegradable polymers PLA, PLGA and/or mixtures, relies on the use for the study and treatment of hypertension, other cardiovascular diseases and their complications (no limitant examples: acute myocardial infarction, stroke, left heart failure, diabetes angipathy, peripheral ischemia, angina and progression of the heart failure after a myocardial infarction and atherosclerosis) tumors, diabetes melitus, sperm motility and spermatogenesis blocking, nephropathies, sexual impotence, gastrointestinal and gynecologic disorders, angiogenesis, hair loss, blood diseases and angioplasty (post-angioplasty restenosis, endovascular protese) in hot-blood animals.

The pharmaceutical compositions claimed therein comprises the Evasins included in cyclodextrins or their derivatives, or Evasins their analogues and derivatives associated or included in carriers and/or excipients pharmaceutically acceptable, alone or mixed or associated at least with one pharmacologically active agent, or Evasins their analogues and derivatives included or not in cyclodextrins, microencapsulated or not in controlled-release systems such us the liposomes and PLA, PLGA biodegradable polymers and/or mixtures. The present invention also comprises the identification of other biochemical mechanisms of action of Evasins having an application in the study and treatment of chronic degenerative diseases.

The pharmaceutical compositions of the Evasins, and their analogues and derivatives, except the Evasin 7a, there in are characterized for presenting differential inhibitory activity for the neutral endopeptidase (Ki in the micro-molar range) and the Angiotensin I converting enzyme (Ki—in the nano-molar range). Another characteristic of these pharmaceutical compositions and formulations is the increase of the biodisponibility, duration and/or efficacy of the cited peptide effect when included in cyclodextrins, for example in a oral formulation.

The present invention may be better understood based on the following no limitant examples.

### EXAMPLE 1

### Synthesis, Purification and Characterization and Enzymatic Assay to Determine the Selective Inhibition of Catalitics Site of the Angiotensin I Converting Enzyme by Evasin

This example describe the synthese, purification, characterization and site C or N-terminal selective inhibition of the ECA by Evasins. The oligopeptides were synthesized according to the state of art of the methodology. The strategy Boc is based on the data described in Barany, G. & Merrifield, R. B. (Gross, E. & Meinhofer, J., Eds.) (1980), The Peptides: Analysis, Synthesis and Biology, vol. II, 1, Academic Press, New York. 2-Stewart, J. M. & Young, J. D. (1984), Solid Phase Peptide Synthesis, Pierce Chemical Company, Rockford.

The oligopeptides were modified according to a methodology of the state of the art. The proposed changes were referred and exemplified in the review of Victor J. Hruby published in the journal Nature, 1, 847-858, 2002.

The synthetic Evasins were purified in a HPLC system and the eluded material was analysed by mass spectrometry.

In the purification the solvents utilized were all of the HPLC grade, degree) and the water utilized was obtained by distillation and filtration in the Milli-Q system, equipped with to cartridge to retain salt and the organic compound.

The fractions from purification were submitted to mass spectrometry analysis to conforming the Evasins molar mass after purification.
The enzyme assays for the recombinant ACE inhibition were conducted using the substrate Mca-Ala-Ser-Asp-Lys-DpaOH, at 25° C., in a 50 mM Hepes buffer (pH 6.8), 200 mM NaCl and 10 PM ZnCl₂. The reactions were continuously monitored by determining the fluorescence increase at λₑₘ=390 nm (λₑₓ=340 nm), provided by the substrate cleavage (S=Kₘ, 40 μM by ACE in a fluorometer. The Evasins were pre-incubated with the enzyme before the substrate addition.

TABLE 1
Assay of the inhibition of the activity of the C and N-terminal catalytic
site of the Angiotensin I converting enzyme by the Evasins.

ECA (nM) - Ki

| PEPTIDE | C-terminal | N-terminal |
|---|---|---|
| EVASIN-5a | 1280 | 399 |
| EVASIN-7a | 40000 | 70000 |
| EVASIN-9a | 1 | 100 |
| EVASIN-10a | 33 | 130 |
| EVASIN-10c | 0.5 | 200 |
| EVASIN-11b | 57 | 3000 |
| EVASIN-11c | 40 | 2000 |
| EVASIN-11e | 300 | 100 |
| EVASIN-12a | 25 | 25 |
| EVASIN-12b | 150 | 5 |
| EVASIN-13a | 50 | 50 |

The results of Kᵢ values for the ACE inhibition by Evasins were in the order of nM. Most of the Evasins were selective inhibitors for the C-terminal site, Evasin-10c, for example, showed a Kᵢ value of 0.5 nM, that is, 400 times more selective for this dominium. While other Evasins showed selective inhibition for the N-terminal site, for example, the Kᵢₛ values obtained using Evasin-12b were 5 nM and 150 nM for the N- and C-terminal sites, respectively.

Considering the state of the art, it was demonstrated the mice with Angiotensin I converting enzyme genetic deletion are infertile (.(Esther C R Jr, Howard T E, Marino E M, Goddard J M, Capecchi A M Bernstein K E. Mice lacking angiotensin-converting enzyme have low blood pressure, renal pathology, and reduced male fertility. Lab Invest. 1996 May; 74(5):953-65) and the testicular ACE corresponds to the ACE somatic N-terminal site (Ramaraj P, Kessler S P, Colmenares C, Sen G C Selective restoration of male fertility in mice lacking angiotensin-converting enzymes by sperm-specific expression of the testicular isozyme. J Clin Invest. 1998 Jul. 15; 102(2):371-8), Pharmaceutical compositions of the Evasin, their analogues and derivatives have the potential to be utilized as male contraceptive. Thus, the present invention also is characterized by the mixture of cyclodextrins organic-aqueous or solid solutions or cyclodextrins derivatives from the alquil, hydroxialquil, hydroxipropil e acyl group with cross bond or cyclodextrins polymers with organic-aqueous or solid solutions of the Evasins and/or of its analogues to be used as a male contraceptive. On the other hand, the Evasins with the selectivity to the C-terminal site may present distinct characteristic different from the ones presented in the inhibitors not selectives or selectives to the N site.

### EXAMPLE 2

### Enzyme Assays for Determining the Evasins—Induced NEP Inhibition

The fluorometric assays for the recombinant NEP inhibition were conducted using the substrate Abz-RL-EDDnp in a 50 mM Tris-HCl buffer, pH 7.5, at 37° C. The reactions were continuously monitored by determining the fluorescence increase at % m=418 nm (sex=318 nm), provided by the substrate cleavage (Kₘ=8.4 μM) by NEP in a fluorometer. The Evasins were pre-incubated with the enzyme before the substrate addition.

The Evasins are not very potent NEP inhibitors, the results of Kᵢ values for the Evasins-induced NEP inhibition were in the order of μM. One of the best inhibitors was Evasin-9a, that showed a Kᵢ value of 86 μM.

TABLE 2
Assay of the neutral endopeptidase activity
inhibition by the Evasin action

| PEPTIDE | NEP (μM) - Ki |
|---|---|
| EVASIN-5a | 550 |
| EVASIN-7a | 492 |
| EVASIN-9a | 86 |
| EVASIN-10c | 253 |
| EVASIN-11b | 476 |
| EVASIN-11c | 589 |
| EVASIN-12a | 106 |
| EVASIN-12b | 150 |
| EVASIN-13a | 473 |

The inhibition constants (Kᵢ) values were determined by means of the ratio of the apparent inhibition constant (Kᵢ₍ₐₚₚ₎) and the substrate Kₘ (Salvesen and Nagase, 1990). In: Proteolytic enzymes a practical aproach, Beynon and Bond Eds Oxford University Press, England, 87-88).

The results above show the low affinity of the same by NEP, suggesting a possible decrease of the collateral effects such as angioeodema and cough when compared to the vasopeptidases inhibitors (omapatrilat for example) related in the state of the technique.

### EXAMPLE 3

### Evasins Brady Potentiating Activity Test—Biological Assay in Guinea Pig Isolated Ileum

The potentiating activity of synthetic peptides using the smooth muscles contractile activity induced by bradykinin was tested and the UP values were determined using the guinea pig isolated ileum preparation. The UP corresponds to the Evasin concentration (nmol/mL of preparation) that is able to change the response effect of a single bradykinin dose into an equivalent double dose.

Female guinea pigs were used. Before starting the assays, the ileum was kept in a Tyrode solution. Next, one of the ends of the ileum segment, measuring 1.5 to 2.0 cm, was tied up to a semi-ring contained in the bottom of a glass cup containing Tyrode saline at 37° C. with constant oxygen bubbling using a capillary; the other end was secured to a previously calibrated lever. The tension maintained was 1 g and the guinea pig isolated ileum contractions were recorded. So as to determine the samples potentiating effect on bradykinin, a log-dose response curve of the bradykinin effect on the guinea pig isolated ileum was plotted. The bradykinin activity is determined by measuring the guinea pig isolated ileum contractions and the potentiating activity is expressed in terms of an increased tissue response to a standard bradykinin dose as per Shimuta et al., Eur. J. pharmacol. 70 (4), 551-554 (1981).

The Evasins were individually tested and spiked before the addition of a single bradykinin dose. The samples dilutions were prepared using deionized water upon use. The measured response was interpolated on the linear portion of the log dose-effect curve, thus obtaining the potentiating activity in terms of an increase in the preparation response for a standard bradykinin dose. TYRODE solution: stock solution I 20 mL, stock solution II 40 mL, diphenydramine solution (1 mg/mL) 1 ml, atropine solution (1 mg/mL) 1 mL, 5.60 mM D-glicose and H₂O q.s. 1 L. All reagents used in this assay were analytical-grade reagents.

All Evasins potentiated the contractile action of bradykinin in guinea pig isolated ileum, duplicating the contractile effect of bradykinin at concentrations ranging from 0.22 to 30 nmols.

### EXAMPLE 4

### Action of Evasins on the Blood Pressure of Anesthetized Rat

The potentiating activity of the bradykinin hypotensive effect was tested in anesthetized rats. Normotensive male rats (WKY) were anesthetized using pentobarbital sodium (Hypnol® Cristália, 50 mg/kg, intraperitoneal) and placed on a controlled-temperature board for maintaining the body temperature between 36.5° C. and 37° C. A polygraph coupled to a physiological transducer was used. The blood pressure ranging values were obtained by integrating the areas limited by the baseline pressure and comparing them to the values obtained from control assays. In in vivo assays, the potentiating activity of Evasins on the hypotensive effect of bradykinin on anesthetized rat blood pressure was observed. Two parameters were determined for comparison of the potentiating effects of bradykinin on the anesthetized rat blood pressure (n=5):

1) Intensity of the potentiating effect of the hypotensive activity of bradykinin on the anesthetized rats blood pressure: This value was defined as the hypotension percentage (%) increase caused by a single dose of bradykinin obtained after the infusion of 200 nmol of potentiator. 2) Potentiating effect length: Time required to reduce the potentiating effect on a single dose of bradykinin by 50%.

The hypotensive effect of bradykinin was potentiated by Evasins in the range from 40 to 340% in anesthetized rats by intravenous injection, at a steady concentration of 200 nmoles of Evasin/rat. A minimum length of 10 minutes was observed and even exceeded 120 minutes for a reduction of the initial potentiating effect by 50%.

TABLE 3
Potentiation by the Evasins of the bradykinin hypontensive
effect on anesthesiated rats blood pressure

| PEPTIDE | Bk potentiation(%) | Duration time |
|---|---|---|
| EVASIN-5a | 102.5 | 10 |
| EVASIN-7a | 41.8 | 120 |
| EVASIN-10c | 195 | 95 |
| EVASIN-11b | 164.3 | 90 |
| EVASIN-11c | 67 | 28 |
| EVASIN-11e | 41 | 35 |
| EVASIN-12b | 340 | 40 |
| EVASIN-13a | 45 | 30 |

### EXAMPLE 5

### Preparation of the Inclusion Compound between β-Cyclodextrins and their Derivatives and the Evasins and their Analogues

This example presents the characterization of the inclusion compound between HP-β cyclodextrin and the evasin 5a, such as no limitant example. The preparation is made in molar ratios of β-cyclodextrin and their derivatives and Evasins and their analogs in aqueous:solutions (1:1 and 1:2). The solutions mixture is subject to constant stirring up to the complete dissolution of β-cyclodextrin. Subsequently, the mixture is frozen at liquid nitrogen temperature and subject to the lyophilization process for 24 hours. The solid thus obtained was characterized using physicochemical analysis techniques. Nuclear magnetic ressonance was the technique providing relevant information on the interaction host/guest.

The preparation was made in equimolar ratios of cyclodextrin and peptides. The table below shows the inclusion compounds so prepared. These systems were subjected to biological tests.

TABLE 4
Inclusion compounds between cyclodextrins and
Evasins that were submitted to biological tests.

| EVASIN | Cyclodextrin |
|---|---|
| EVASIN-5a | β-Ciclodextrina |
| EVASIN-7a | β-Ciclodextrina |
| EVASIN-9a | β-Ciclodextrina |
| EVASIN-10c | β-Ciclodextrina |
| EVASIN-5a | HP-β-CD |
| EVASIN-7a | HP-β-CD |
| EVASIN-9a | HP-β-CD |
| EVASIN-10c | HP-β-CD |
| EVASIN-12b | HP-β-CD |

After the preparation of solutions and the simulation of the NMR ¹H and ¹³C spectra of HB-β-CD and peptides, the NMR-characterization step of the inclusion compound HB-β-CD/BPP-5a, as well as free BPP-5a and HB-β-CD free. Hydrogen—¹H NMR, to structural elucidation of the cyclodextrins and host molecules BPP-5a; COSY, TOCSY, ¹³C-DEPT135, HMQC to structural elucidation of BPP-5a structural; variations analyses of chemical dislocation—δ; time measurements of the longitudinal relaxation—RMN T₁ and NOESY experiments (Nuclear Overhauser Effect Spectroscopy), heading to verify the inclusion. δ and T1 study utilized as a probe only the hydrogens of the host molecules as the cyclodextrin hydrogen signal are overlapped.

To structural elucidation of the pentapeptide BPP-5a to RMN, was utilized as reference, the RMN ¹H spectral of the specifics free aminoacids (THE SADTLER STANDARD SPECTRA. Sadtler Research Laboratories. 1972). Due to a complexity of the molecule, as well as its spectral it was necessary the use of many RNM techniques as one dimensional as bi-dimensional, being also necessary to use 400 MHz spectrometer. The sample was dissolved in D₂O, the spectral based in the hydrogen nuclear magnetic resonance were very simplified, in function of the hydroxy, amida and amina groups hydrogen signals absence, once that they are changed to the deuterium atoms of the solvent.

Based on the corresponding simulations for Evasin-5a, the actual spectrum of the pure compound was analyzed, the following being assigned:
1) The NMR ¹H spectrum, as well as the COSY, showed an intense signal at 4.8 ppm, as a result of the presence of the impurity “H₂O” in the deuterated solvent.
2) The “CH₂” groups hydrogens are next to the water signal; these hydrogens are a link between the peptide bonds and the amino acids functional groups.
3) The “CH₂” groups are present within the region comprised between the chemical displacement at 2.8-3.7 ppm, these groups being bonded to the “CH₂” groups next to the peptide bonds.
4) Between 1.3-2.5 ppm approximately, multiplets referring to “CH₂” that belong to several functional groups can be seen.
5) At 1.2 ppm, a dublet referring to the CH₃ group of the segment of the amino acid Alaline can be seen. Using COSY, the scalar coupling of this group with a “CH” at 4.6 ppm (quartet) can be observed.
6) The region comprised between the chemical displacement range from 7.7 to 7.1 ppm was assigned to the aromatic group spins system (corresponding to the segment of the amino acid Tryptophan).

The HP-β-CD spectrum showed to be highly complex, difficult to assign, even when based on the simulated spectrum. However, it is possible to assign some signals mentioned below:

Between the chemical displacements at 3.3 to 4.3 ppm, a complex multiplet is seen as a result of the “CH₂” groups of carbons bonded to hydroxyls. At the chemical displacement interval between 1.1 to 1.8 ppm, the signals referring to the hydroxypropyl groups can be found.

Concerning the NMR assay of the inclusion compound, comparing the pure Evasin-5a sprectrum, observed a clear separation of spectral lines, with a consequent chemical displacement variation within the aromatic groups region (δ≈7.7-7.1). This is a strong indication of the interaction of the HP-β-CD with this region of this peptide. This phenomenon is due to the electrons no ligants of the atoms of oxygen C1-O1-C4 of the cyclodextrin that can cause a disturbance in the electronic distribution of the aromatic groups of the peptide. This result suggests the possible encapsulation of the aromatic group into the HP-β-CD cavity.

In relation to the Evasin-5a, presented in the table 1 and 2 (values of T1), observed that only the hydrogen 16 and 19 had significant variations, once that the T1 variation of this nucleus is much bigger than the standard deviation obtained in each measurement. The others hydrogens analyses presented T1 variations that are questionable if compared to the standard deviation of each measurement.

Analyzing the tables below, it was observed a reduction of the relaxation time, T1, of the host molecules aromatics hydrogens. This alteration suggests a reduction of the mobility of the guest molecules, a symmetry change and a slow rotational kinetic after the inclusion of the host molecules in the cavity of the cyclodextrin.

TABLE 5
Values of longitudinal relaxation time of some nuclei de
1 H of the peptide BPP-5a pure, measure in 200 MHz.

| Group number | Chemical displacement | Relaxation time (T1) |
|---|---|---|
| 19 | 7.5052 ppm | 0.7603 ± 0.0078 s |
| 16 | 7.4068 ppm | 1.2927 ± 0.0122 s |
| 21 | 4.3592 ppm | 0.4147 ± 0.0343 s |
| 14 | 3.1657 ppm | 0.1778 ± 0.0031 s |
| 24 | 2.8750 ppm | 0.5969 ± 0.0025 s |
| 9 e 10 | 1.8857 ppm | 0.2864 ± 0.0090 s |

TABLE 6
Values of the longitudinal relaxation time of some nuclei of the 1H
of the system HP-^{β}-CD/BPP-5a, measured in 200 MHz

| | Chemical | | |
|---|---|---|---|
| Group number | displacement | T1/s | Δ_{T1/s} |
| 19 | 7.5353 ppm | 0.5342 ± 0.0046 | −0.2261 |
| 16 | 7.4063 ppm | 0.7127 ± 0.0043 | −0.5800 |
| 21 | 4.3760 ppm | 0.3533 ± 0.0670 | −0.0614 |
| 14 | 3.2020 ppm | 0.1518 ± 0.0129 | −0.0260 |
| 24 | 2.8890 ppm | 0.5182 ± 0.0011 | −0.0787 |
| 9 e 10 | 1.6040 ppm | 0.2634 ± 0.0046 | −0.0230 |

It was possible to determine the guest molecules spacial arrangement inside of the cyclodextrins cavity using the NOESY of β-CD e BPP-5a/HP-β-CD experiments, adding to the others RNM data. The NOESY experiment was carried out with D8=650 ms to the BPP-5a. This was the unique value of D8 that observed cross correlations that could indicate a spacial proximity between the HP-β-CD and BPP-5a hydrogens as example no limitant.

### EXAMPLE 6

### Effect of Evasins On The Blood Pressure of Non Anesthetized Hypertensive Rat

One day before the assay, the animals were subjected to a surgery for cannulation of the femoral artery and vein. The rats were anesthetized with ether and placed in dorsal decubitus on a surgical board. A small skin incision was made, thus separating the musculature for locating the femoral vasculonervous bunch. The cannulas were inserted into the inferior vena cava through the femoral vein for administering the drug and into the abdominal aorta through the femoral artery for recording the cardiovascular parameters. After insertion, the cannulas were tied to the bunch using a surgical line. Next, the cannulas were directed subcutaneously with the aid of a trocater to the scapular waist where they were exteriorized and secured using a suture line. The arterial cannula was used for recording the cardiovascular parameters and the venous cannula for administering the drugs.

The blood pressure and the heart rate were recorded one day after the femoral artery and vein cannulation. The assay was conducted with non-anesthetized animals with free movement. The rats pulsed arterial pressure (PAP), the mean arterial pressure (MAP) and heart rate (HR) were monitored by a computer, using a data acquisition system (BIOPAC). The data were collected during all the experiments.

Before the drug administration, the rats PBP, MBP and HR were monitored. After the bolus injection of Evasin in a total volume of 0.2 mL (NaCl 0.9% solution), the resulting hypotensive effect and the effect length were monitored. In this assay, standard doses of each peptide (70 nmol/100 g of body weight) were tested (n=4). Evasin-10c was the most potent peptide with the best hypotensive effect, producing a reduction of −29.5±9.50 mmHg in the mean blood pressure, followed by Evasin-9a with a MBP reduction of −27.0±1.50 mmHg. Evasin-5a and the Evasin-7a produced a maximum MBP reduction of −19.17±4.36 mmHg and 13.00±2.78 mmHg respectively.

When comparing the effect length, it was observed that Evasin-7a was the peptide showing a hypotensive effect lasting more than 100 minutes. Evasin-9a and Evasin-10c were similar in terms of effect length of 84 and 94 minutes, respectively. The effect length of Evasin-5a was short; 37 minutes after the administration the MAP returned to the baseline values.

### EXAMPLE 7

### Effect of Encapsulated Evasins in Cyclodextrins on The Blood Pressure of Awake Hypertensive Rat

This example describes the inclusion compound administration effect between the Evasin-5a and hydroxiprolil-α-cyclodextrina BPβCD) on the blood pressure of the hypertensive rats. One day before the assay, the animals were subjected to a surgery for cannulation of the femoral artery and vein. The rats were anesthetized with ether and placed in dorsal decubitus on a surgical board. A small skin incision was made, thus separating the musculature for locating the femoral vasculonervous bunch. The cannulas were inserted into the inferior vena cava through the femoral vein for administering the drug and into the abdominal aorta through the femoral artery for recording the cardiovascular parameters. After insertion, the cannulas were tied to the bunch using a surgical line. Next, the cannulas were directed subcutaneously with the aid of a trocater to the scapular waist where they were exteriorized and secured using a suture line.

The blood pressure and the heart rate were recorded one day after the femoral artery and vein cannulation. The assay was conducted with non-anesthetized animals with free movement. The animals pulsed arterial pressure (PAP), the mean arterial pressure (MAP) and heart rate (HR) were monitored by a computer, using a data acquisition system (BIOPAC). The data were collected during all the experiments.

Before the drug administration, the rats PBP, MBP and HR were monitored during 60 minutes. After the bolus injection of Evasin in a total volume of 0.2 mL (NaCl 0.9% solution), the resulting hypotensive effect and the effect length were monitored. A standard doses of 0.071 nmol/100 g of body weight, n=6, was utilized in this assay.

When comparing the results obtained from the administration of free Evasin-5a and the Evasin-5a included in HPβCD, no differences were observed in the maximum hypotensive effect between the administration of the free pentapeptide or in the encapsulated form, reduction of MAP 23±4.2 mmHg and 22±3.3 mmHg, respectively. We observed a relevant difference in the effect length, where the encapsulated Evasin-5a was able to increase the free peptide effect length by more than 4 times than the duration time of the free peptide, 140 and 38 minutes respectively.

### EXAMPLE 8

### Comparative Effect of the Evasins on the Hypertensive Rats Awaked Blood Pressure (SHR And TGR(mREN2)L27

This example, no limitant, describes the infusion effect of the Evasin-5a on hypertensive rats blood pressure of different strain.

One day before the assay, the animals were subjected to a surgery for cannulation of the femoral artery and vein. The rats were anesthetized with ether and placed in dorsal decubitus on a surgical board. A small skin incision was made, thus separating the musculature for locating the femoral vasculonervous bunch. The cannulas were inserted into the inferior vena cava through the femoral vein for administering the drug and into the abdominal aorta through the femoral artery for recording the cardiovascular parameters. After insertion, the cannulas were tied to the bunch using a surgical line. Next, the cannulas were directed subcutaneously with the aid of a trocater to the scapular waist where they were exteriorized and secured using a suture line.

The blood pressure and the heart rate were recorded one day after the femoral artery and vein cannulation. The assay was conducted with non-anesthetized animals with free movement. The animals pulsed arterial pressure (PAP), the mean arterial pressure (MAP) and heart rate (HR) were monitored by a computer, using a data acquisition system (BIOPAC). The data were collected during all the experiments.

Before the drug administration, the rats PBP, MBP and HR were monitored during 60 minutes. During and after the infusion of the Evasin 5a, 900 μg/100 g corporal weight/hour in NaCl solution 0.9% (n=6), the cardiovascular parameters were registered. The administration of the evasin-5a in both strains produced the same decreased of the MAP 28±2.7 mmHg and 34±0.9 mmHg in the SHR and TGR (mREN2) L27 respectively. However, it was observed that the relevant difference in time duration effect after the infusion interruption was over 6 hours in the SHR and only 1 hour in the TGRs. These differences indicate important variations of the biochemical mechanisms of the Evasins 5a action with different degree for the renin angiotensin system activation.

### EXAMPLE 9

### Effect of Acute Administration of the Evasins on SHR Blood Pressure Measured by Telemetry System

A telemetry system was used for measuring the systolic and diastolic pressure, the mean blood pressure and the heart rate. This monitoring system consists in an implantable radio frequency device, a receptor board, a matrix and a computer with a software for data acquisition and analysis (Braga, et al., 2002).

Male SHR rats were used. The animals remained fasted for 24 hours before the surgery. Under anesthesia using 2.5% 2,2,2-tribromoethanol (1 mL/100 g of body weight), the rats were placed in dorsal decubitus on a surgical board, the ventral abdominal region was clipped and aseptically cleaned with iodated alcohol. An incision of about 2 cm in the median abdominal line was made so as to have a good view of the iliac bifurcation area. The bowels were removed so as to allow a complete access to the abdominal blood vessels. The adipose and connective tissues along the vascular bed were delicately removed with the aid of cotton swabs and gauzes until the abdominal aorta could be identified and properly isolated from the vena cava. A cordonet thread wetted with saline embraced the aorta so as to prevent the blood flow and a small cut was made using the needle (25×8) bent in an angle of 90°. Next, using a guide pincer, the device polyethylene catheter received the biocompatible gel and was inserted into the artery. The catheter inlet area was cleaned and dried and a tiny amount of tissue glue was applied. Over the glue, a small piece of cellulose paper was placed to secure the catheter in the aorta. The device battery was started with the magnet and, using an AM radio (not tuned), the typical sound of the catheter position in the aorta was recorded. The abdominal musculature was sutured, securing the device by its silicone tab. Next, the skin was sutured. Asepsis was made using iodated alcohol and 0.1 mL of pentabiotic was then administered intramuscularly. The animals were placed in individual cages and remained under warmed conditions until totally recovered from anesthesia. After recovery, the animals were taken to the telemetry room climatized at 25° C. and maintained on a 12 hr/12 hr light/dark cycle (6:00 a.m. to 6:00 p.m. day and 6:00 p.m. 6:00 am. night). Water and food were given ad libitum.

Before proceeding to the assay, the animals were placed in individual cages (15 cm×12 cm×6 cm) and kept for 8 days until the telemetry plots indicated blood pressure and heart rate recovery. The data was sampled every 10 minutes for 10 seconds/24 hours.

The results obtained, by oral administration, of the Evasin-7a and Evasin-10c, encapsulated in β-cyclodextrin gavage, showed that these two peptides exert a hypotensive activity in spontaneously hypertensive rats. Evasin-7a showed a length of 9 hours, with a maximum reduction of the MAP of 20 mmHg after 5 hours and within 8 mmHg after the peptide administration and 5 hours, respectively. Evasin-10c had a maximum hypotensive effect of 5 hours and a maximum reduction of 13 mmHg, two hours after administration.

### EXAMPLE 10

### Biodistribution Of Evasins

The intravenous administration of ¹²⁵I labeled Evasins according to the chloramine T method in mice showed that these peptides are noticeably concentrated in the kidneys. For example no limitant, the intravenous injection of ¹²⁵I-Evasin10c showed, after 15 minutes, that this peptide had a concentration about twice as high (per gram of tissue) in the kidneys than in the lungs and liver. In other tissues and blood, the radioactive peptide concentration was significantly lower. This difference increases in the kidneys when compared to other tissues, peak concentrations are reached after 30 minutes, drop quickly in tissues and much slower in the kidneys where they remain at about 50% of the peak concentration observed 3 hours after the administration. This same distribution profile was seen when the radioactive peptide was administered together with captopril at a molar concentration 10 times higher than the peptide concentration, reducing about 30% of the peak concentration reached by the radioactive peptide when compared to that reached without captopril.

Both the biodistribution and the dwelling time of Evasins in the kidneys showed that they have a greater selectivity for the kidneys and remain bonded to that tissue for a longer period of time than captopril. To the other hand, the 30% reduction of the renal binding of the Evasins are ligands to the angiotensin converting enzyme.

1-31. (canceled)

32. Pharmaceutical compositions of the peptides, secreted by the snake venom glands particularly Bothrops Jararaca, vasopeptidases inhibitors, Evasins, their analogues and derivatives characterized for comprising:
a) oligopeptides of 5-13 amino acids
EVASIN-5, SEQ ID No. 104 to SEQ ID No. 103,
EVASIN-6, SEQ ID No. 104 to SEQ ID No. 243,
EVASIN-7, SEQ ID No. 244 to SEQ ID No. 325,
EVASIN-9, SEQ ID No. 326 to SEQ ID No. 422 and SEQ ID No. 595,
EVASIN-10, SEQ ID No. 423 to SEQ ID No. 516,
EVASIN-11, SEQ ID No. 517 to SEQ ID No. 556,
EVASIN-12, SEQ ID No. 557 to SEQ ID No. 589,
EVASIN-13, SEQ ID No. 590 to SEQ ID No. 594;
b) inclusion compounds of the Evasins, their analogues or derivatives in cyclodextrins or their derivatives, or associated or included in carriers and/or excipients acceptable pharmaceutically, alone or mixed or associated at least with another active pharmacological agent;
c) the Evasins, SEQ ID No. 1 to SEQ ID No. 595, their analogues and derivatives included or not in cyclodextrins microencapsulated or not in controlled-release systems such as liposomes and biodegradable polymers and/or mixtures.

33. Pharmaceutical compounds of the Evasins, their analogues and derivatives characterized by utilizing the Evasins 7a, SEQ ID No. 253, EVASIN-10c, SEQ ID No. 472, 11e, SEQ ID No. 555, 12b, SEQ ID No. 557, their analogues and derivatives as a molecular model for development of drugs and/or formulations based on peptides compounds and/or non-peptide vasopeptidase inhibitors.

34. Pharmaceutical compositions of the Evasins, SEQ ID No. 1 to SEQ ID No. 595, their analogues and derivatives characterized by utilizing the Evasins 7a, SEQ ID No. 253, EVASIN-10c, SEQ ID No. 472, 11e, SEQ ID No. 555, 12b, SEQ ID No. 557, their analogues and derivatives as a molecular model for development of drugs and/or formulations based in peptides compound and/or non-peptide ligand agonists and antagonists of the angiotensin converting enzyme bound to the membrane.

35. Compositions of the Evasins, their analogues and derivatives, except the Evasins 7a, SEQ ID No. 253, according to claim 32 characterized for presenting differential inhibitory activity for the neutral endopeptidase (Ki in the micro molar range) and the angiotensin I converting enzyme (Ki in the nano molar range).

36. Pharmaceutical compositions of the Evasins, their analogues and derivatives according to claim 32 characterized for presenting selective inhibitory activity for the C-terminal domain of the angiotensin I converting enzyme, being 50a 400 fold more potent for the C-domain than for the N-domain.

37. Pharmaceutical compositions of the Evasins, their analogues and derivatives according to claim 32 characterized for presenting selective binding to the C-terminal domain of the angiotensin I converting enzyme, being 50 a 400 fold more potent to the C domain than for the N-domain.

38. Pharmaceutical compositions to the Evasin-7a, SEQ ID No. 253, their analogues and derivatives according to claim 32 characterized for presenting similar inhibitory activity similar to the neutral endopptidase and the angiotensin I converting enzyme.

39. Utilization of the Evasins 7a, SEQ ID No. 253, EVASIN-10c, SEQ ID No. 472, 11e, SEQ ID No. 555, 12b, SEQ ID No. 557, their analogues and derivatives as a molecular model to the development of drugs and/or formulations based on peptide compounds and/or non-peptide compounds characterized for presenting vasodilator and/or vasoprotector activity.

40. Pharmaceutical compositions of the Evasins, their analogues and derivatives according to claim 32 for application in the study and treatment of arterial hypertension and others cardiovascular diseases and their complications characterized by the use of inclusion compounds or association of the Evasins, their analogues and derivatives with the cyclodextrin and their derivatives, microencapsulated or not in controlled-release system such as example, liposome and the biodegradable polymers, and/or mixtures.

41. Pharmaceutical compositions of the Evasins, their analogues and derivatives according to claim 32 useful for the study and treatment of acute myocardial infarction, left ventricular hypertrophy diabetic, vasculopathy, peripheral ischemia, angina, progressive heart failure after a myocardial infarction, atherosclerosis, tumors, diabetes melitus, sperm motility, blockade of spermatogenesis, nephropathies, sexual disfunction, gastrointestinal and gynecologic disorders, angiogenesis, hair loss, blood diseases, angioplasty (restenosis after-angioplasty, endoluminal protese) in warm-blood animals characterized by the use of inclusion compounds or association of the Evasins, their analogues and derivatives with cyclodextrins and their derivatives, microencapsulated or not in controlled-release systems such as the liposome and the biodegradable polymers and/or mixtures.

42. Pharmaceutical compositions according to claim 32 characterized by the mixture of the organic-aqueous solids or solutions of cyclodextrins or derivatives of cyclodextrins selected from the group containing alkyl, hydroxyalkyl, hydroxyalkyl, hydroxypropyl and acyl or cyclodextrins with cross-bonds or cyclodextrins polymers with solutions of Evasins, their analogues and derivatives at a molar ratio of 1:1 or 1:2.

43. Pharmaceutical compositions of the SEQ ID No. 1 to SEQ ID No. 595, their analogues and derivatives characterized by the use of the cyclodextrins, or others controlled-release systems including, liposomes, biodegradable polymers derivatives of biodegradable polymers or mixture of these systems.

44. Utilization of the Evasins SEQ ID No. 1 to SEQ ID No. 595, their analogues and derivatives as molecular models for the development of drugs and/or formulations with differential inhibitory activity for the neutral endopeptidase and the angiotensin I converting enzyme according to the claim 35, characterized for presenting a lower inhibitory activity for the neutral endopeptidase and consequently with a smaller possibility of incidence of collateral effects such as cough and angiodema.

45. Pharmaceutical compositions for the study and treatment of arterial hypertension and others cardiovascular diseases and their complications characterized by the mixture of organic-aqueous or solid solutions of cyclodextrins or derivatives of cyclodextrin selected from the groups containing alquil, hydroxialquil, hydroxipropil and acyl or cyclodextrins with crossed bonds or polymers of cyclodextrins, with aqueous or solids solutions of Evasins SEQ ID No. 1 to SEQ ID No. 595, their analogues and derivatives.

46. Pharmaceutical compositions to study and treatment of the acute myocardial infarction, left ventricular hypertrophy diabetic vasculopathy, peripheral ischemia, angina, progressive heart failure after a myocardial infarction, atherosclerosis, tumors, diabetes melitus, sperm motility, blockade of spermatogenesis, nephropathies, sexual disfunction, gastrointestinal and gynecologic disorders, angiogenesis, hair loss, blood diseases, angioplasty (restenosis after-angioplasty, endoluminal protese) in warm-blood animals characterized by mixture of organic-aqueous or solid solutions of cyclodextrins or derivatives of cyclodextrins selected from the group containing alkyl, hydroxyalkyl, hydroxypropyl and acyl or cyclodextrins with crossed bonds or polymers of cyclodextrins at a molar ratio of 1:1 or 1:2 with aqueous or solid solutions of Evasins SEQ ID No. 1 to SEQ ID No. 595, their analogues and derivatives.

47. Pharmaceutical compositions to be used as male contraceptive characterized by the mixture of organic-aqueous or solid solutions of cyclodextrins or derivatives of cyclodextrins selected from the group containing alkyl, hydroxyalkil, hydroxypropyl and acyl or cyclodextrins with crossed bonds or polymers cyclodextrins at a molar ratio of 1:1 or 1:2 with aqueous or solid solutions of Evasins SEQ ID No. 1 to SEQ ID No. 595, their analogues and derivatives.

48. Pharmaceutical compositions of the Evasins, their analogues and derivatives according to claim 32, characterized by the increase of the biodisponibility of the cited Evasins when included in cyclodextrins or their derivatives, or associated or included in carriers and/or excipients pharmaceutically acceptable, alone or mixed.

49. Pharmaceutical compositions of the Evasins, their analogues and derivatives according to claim 32 characterized by the increase of the duration and/or efficacy of the Evasins effect, their analogues and derivatives when included in cyclodextrins or their derivatives, or associated or included in carriers and/or excipients pharmaceutically acceptable, alone or mixed.

50. Oral pharmaceutical compositions of the Evasins, their analogues and derivatives, according claim 32 to be use in the treatment of hypertensive emergency characterized by the used of the mixture with excipients pharmaceutically acceptable including water, saline solution, buffer solutions, Ringer solution, dextrose solution, Hank solution, Biocompatible saline solutions, containing or not polyethilene glycol.

51. Oral pharmaceutical compositions of the Evasins, its analogues and derivatives according to claim 32 characterized by the increase of the biodisponibility of the cited Evasins when included in cyclodextrins or their derivatives, or associated or included in carriers and/or excipients pharmaceutically acceptable, alone or mixed.

52. Pharmaceutical compositions oral of the Evasins, their analogues and derivatives according to claim 32 characterized by the increase of the duration and/or efficacy of the Evasins effect, their analogues and derivatives when included in cyclodextrins or their derivatives, or associated or included in carriers and/or excipients pharmaceutically acceptable, alone or mixed.

53. Compositions and formulations for intramuscular, subcutaneous, topic, inhalatory (pulmonary, intranasal, intramouth) administration or as device that can be implanted or injected, of the Evasins, their analogues and derivatives according to claim 32 characterized by the increase of the biodisponibility of the cited Evasins, their analogues and derivatives when included in cyclodextrins or their derivatives, or associated or included in carriers and/or excipients pharmaceutically acceptable, alone or in mixture.

54. Compositions and formulations for intramuscular, subcutaneous, topic, inhalatory (pulmonary, intranasal, intramouth) administration or as device that can be implated or injected, of the Evasins, their analogues and derivatives according to claim 32 characterized by the increase of the biodisponibility of the cited Evasins, their analogues and derivatives when included in cyclodextrins or their derivatives, or associated or included in carriers and/or excipients pharmaceutically acceptable, alone or in mixture or mixed or associated at least with another agent pharmacologically active microencapsulated or not in controlled-release systems such as liposome and the biodegradable polymers and/or mixtures.

55. Compositions and formulations for intramuscular, subcutaneous, topic, inhalatory (pulmonary, intranasal, intramouth) administration and as device that can be implanted or injected, of the Evasins, their analogues or derivatives according to claim 32 characterized by the increase of the duration and/or efficacy of the cited Evasins, their analogues or derivatives when included in cyclodextrins or their derivatives, or associated or included in carriers and/or excipients pharmaceutically acceptable, alone or mixed.

56. Compositions and formulations for intramuscular, subcutaneous, topic, inhalatory (pulmonary, intranasal, intramouth) administration or as device that can be implated or injected, of the Evasins, their analogues and derivatives used in the study and treatment of the arterial hypertension and others cardiovascular diseases and their complications according to claim 32, characterized by the use of the Evasins their analogues and derivatives when included in cyclodextrins or their derivatives, or associated or included in carriers and/or excipients pharmaceutically acceptable, alone or in mixture associated, at least, with an additional pharmacologically active agent microencapsulated or not in controlled-release systems such as liposomes and biodegradable polymers and/or mixtures thereof.

57. Compositions and formulations for intramuscular, subcutaneous, topic, inhalatory (pulmonary, intranasal, intramouth) administration or as device that can be implanted or injected, of the Evasins, their analogues and derivatives used in the study and treatment of the acute myocardial infarction, left ventricular hypertrophy, diabetic vasculopathy, peripheral ischemia, angina, progressive heart failure after a myocardial infarction, atherosclerosis, tumors, diabetes melitus, sperm motility, blockade of spermatogenesis, nephropathies, sexual disfunction, gastrointestinal and gynecologic disorders, angiogenesis, hair loss, blood diseases, angioplasty (restenosis after-angioplasty, endoluminal protese) in warm-blood animals according to claim 32 characterized by the use of the Evasins their analogues and derivatives when included in cyclodextrins or their derivatives, or associated or included in carriers and/or excipients pharmaceutically acceptable, alone or in mixture associated, at least, with an additional pharmacologically active agent microencapsulated or not in controlled-release systems such as liposomes and biodegradable polymers and/or mixtures, thereof.

58. Pharmaceutical compositions for intramuscular, subcutaneous, topic, inhalatory (pulmonary, intranasal, intramouth) administration or as device that can be implated or injected, of the Evasins, their analogues and derivatives according to claim 32 characterized by the increase of the biodisponibility of the cited Evasins, their analogues and derivatives when included in cyclodextrins or their derivatives, or associated or included in carriers and/or excipients pharmaceutically acceptable, alone or mixed or associated at least with an additional pharmacologically active agent microencapsulated or not in controlled-release systems such as liposome and the biodegradable polymers and/or mixtures.

59. Pharmaceutical compositions and formulations for intramuscular, subcutaneous, topic, inhalatory (pulmonary, intranasal, intramouth) administration or as device that can be implanted or injected, of the Evasins, their analogues and derivatives according to claim 32, characterized by the increase of the duration and/or efficacy of the Evasins effect, their analogues and derivatives when included in cyclodextrins or their derivatives, or associated or included in carriers and/or excipients pharmaceutically acceptable, alone or mixed

60. Pharmaceutical compositions, intramuscular, intravenous, subcutaneous, topic, inhalation (pulmonary, intranasal, intramouth) or as device that can be implanted or injected, of the Evasins, their analogues structural and conformational according to claim 32 characterized by the increase of the duration and/or efficacy of the Evasins, their analogues and derivatives effect when included in cyclodextrins or their derivatives, or associated or included in carriers and/or excipients pharmaceutically acceptable, alone or mixed, at least, with an additional pharmacologically active agent and/or microencapsulated or not in controlled-release systems such as liposomes and biodegradable polymers and/or mixtures, thereof.

61. Pharmaceutical compositions for intramuscular, intravenous, subcutaneous, topic, inhalation (pulmonary, intranasal, intramouth) administration or as device that can be implanted or injected, of the Evasins, their analogues and derivatives used in the study and treatment of arterial hypertension and others cardiovascular and their complications according to claim 32, characterized by the use of the Evasins their analogues and derivatives when included in cyclodextrins or their derivatives, or associated or included in carriers and/or excipients pharmaceutically acceptable, alone or in mixture associated at least with an additional pharmacologically active agent microencapsulated or not in controlled-release systems such as the liposomes and biodegradable polymers and/or mixtures, thereof.

62. Pharmaceutical compositions for intramuscular, intravenous, subcutaneous, topic, inhalation (pulmonary, intranasal, intramounth) or as device that can be implanted or injected, of the Evasins, their analogues and derivatives used in the study and treatment of the acute myocardial infarction, stroke, left ventricular hypertrophy, diabetic vasculopathy, peripheral ischemia, angina, progressive heart failure after a myocardial infarction, atherosclerosis, tumors, diabetes melitus, sperm motility, blockage spermatogenesis, nephropathies, sexual impotence, gastrointestinal and gynecologic disorders, angiogenesis, hair loss, blood diseases, angioplasty (restenosis after-angioplasty, endoluminal protese) in warm-blood animals according to claim 32 characterized by the use of the Evasins their analogues and derivatives when included in cyclodextrins or their derivatives, or associated or included in carriers and/or excipients pharmacologically acceptable, alone or in mixture associated, at least, with an additional pharmacologically active microcapsulated or not in controlled-release systems such as the liposomes and biodegradable polymers and/or mixtures thereof.
